(19) United States
(12) Patent Application Publication
(10) Pub. No.: US 2010/0092459 A1
(43) Pub. Date: Apr. 15, 2010

(54) Protein Complex and Uses

(75) Inventors: Michael Julian Seckl, London (GB); David Julian Downward, London (GB); Olivier Rafi Emmanuel Pardo, London (GB)

Correspondence Address: SULLIVAN & WORCESTER LLP, 1666 K Street NW, Washington, DC, 20006, US

(73) Assignee: IMPERIAL INNOVATIONS LIMITED, London (GB)

(21) Appl. No.: 12/520,206

(22) PCT Filed: Dec. 22, 2006
(86) PCT No.: PCT/GB06/04894

§ 371 Date: Dec. 28, 2009

(51) Int. Cl. A61K 39/395; C12N 9/12; C07K 16/18; G01N 33/53; A61K 31/7105; A61K 31/711; A61P 35/00

(52) U.S. Cl. 424/130.1; 435/194; 530/387.1; 435/7.2; 435/7.1; 514/44 R; 514/44 A; 435/7.23

(57) ABSTRACT

The present invention relates to a complex comprising two or more of the proteins S6K2, PKCε and B-Raf. The invention also relates to antibodies that specifically bind to the complex, inhibitors of the complex and uses of the antibodies, inhibitors and complex in diagnosing and preventing chemoresistance in a patient.

The present invention relates to a complex comprising two or more of the proteins S6K2, PKCε and B-Raf. The invention also relates to antibodies that specifically bind to the complex, inhibitors of the complex and uses of the antibodies, inhibitors and complex in diagnosing and preventing chemoresistance in a patient.

Cancers are often treated using chemotherapy, which is the use of one or more chemical substances, such as a cytotoxic drug, to “kill” the cancer cells. However, some cancers can develop resistance to these drugs, making their treatment more difficult, or in some cases, impossible. For example, patients with small cell lung cancer (SCLC) often die because of chemoresistance. Small Cell Lung Cancer (SCLC) represents 20% of all lung tumours. Despite initial sensitivity to therapy, relapse with chemoresistant disease is rapid and overall survival is very poor. Chemoresistance may also occur in other cancers, such as non-small cell lung cancer, breast cancer, ovarian cancer and pancreatic cancer.

Therefore, there exists an urgent need for preventing or reversing this chemoresistance in cancer cells.

Growth factors can provide pro-survival signals and in particular, fibroblast growth factor-2 (FGF-2) has been implicated in driving chemoresistance in cancers including SCLC (Pardo et al., 2002; Pardo et al., 2003). Moreover, elevated serum concentrations of FGF-2 is an independent prognostic factor for adverse outcome in SCLC (Ruotsalainen et al., 2002). FGF-2 induces the activation of the extracellular regulated kinase signalling pathway (MEK/ERK) thereby triggering resistance to etoposide (Pardo et al., 2002; Pardo et al., 2003), a drug commonly used in the treatment of SCLC. The pro-survival effect occurred via increased translation of the anti-apoptotic molecules Bcl-2, Bcl-X_{L}, XIAP and cIAP1 (Pardo et al., 2002; Pardo et al., 2003). Fibroblast growth factor-2 (FGF-2) increases the expression of antiapoptotic proteins, XIAP and Bcl-X_{L}, and triggers chemoresistance in SCLC cells.

The 40s ribosomal protein S6 is a component of the 40s subunit of eukaryotic ribosomes. The S6 protein is phosphorylated in response to certain cellular signalling events, such as hormone or growth factor induced proliferation, by two S6 kinases.

Ribosomal S6 kinases, S6K1 and S6K2 also known as S6Kα and S6Kβ (Gout et al., 1998; Lee-Frumen et al., 1999; Shima et al., 1998), both regulate the translational machinery (Dufner and Thomas, 1999). Each kinase has a cytoplasmic and nuclear form but most work has focused on the cytoplasmic proteins, which for simplicity we refer to here as S6K1 and S6K2. They were thought to have overlapping functions as they both phosphorylate the S6 protein. However, recent data suggests that their substrates and roles may be distinct although the precise function of S6K2 is still unclear (Richardson et al., 2004; Valovka et al., 2003). Thus, despite high homology, they differ substantially in their N- and C-terminal domains; S6K1 knock-out mice are small despite increased expression levels of S6K2 (Shima et al., 1998), while S6K2 null mice have no obvious phenotype (Pende et al., 2004); the activation of S6K1 is insensitive to MEK inhibition, but the inventors and others have shown that S6K2 is a novel target of MEK signalling (Martin et al., 2001; Pardo et al., 2001; Wang et al., 2001). WO 00/08173 described the identification of S6K2 and its function as a kinase that phosphorylates the ribosomal S6 protein in vitro.

S6K2 is also regulated by protein kinase C (PKC) (Valovka et al., 2003), a family of proteins involved in the activation of MEK/ERK in several cell systems including SCLC cells (Kawauchi et al., 1996; Seufferlein and Rozengurt, 1996; Zou et al., 1996). The PKC family comprises classical (cPKCs: PKCα, PKCβ, PKCγ), non-classical (nPKCs: PKCδ, PKCε, PKCη and PKCθ) and atypical (aPKCs: PKCζ and PKC₁/λ) classes. While the activation of cPKCs is both Ca²⁺ and phorbol ester dependent, nPKCs only require phorbol esters and aPKC are independent of both agents (Way et al., 2000). Depending on the stimulus used, distinct subclasses of PKC lead to different physiological effects (Way et al., 2000). PKCε can mediate pro-survival/chemoresistance in lung cancer cells (Ding et al., 2002), but the signalling mechanism underlying this effect was not identified. Raf-1 and/or B-Raf are involved in growth factor receptor coupling to MEK/ERK and PKC (Cheng et al., 2001; Hamilton et al., 2001).

The inventors have unexpectedly found that PKCε, B-Raf and S6K2 form a signalling complex in response to FGF-2 treatment. Down-regulation of PKCε induces, whilst PKCε over-expression protects, SCLC cells from drug-induced cell death. This surprisingly correlates with increased S6K2, but not S6K1, activity. Increased S6K2, but not S6K1 kinase activity also enhances cell survival, and downregulation of S6K2, but not S6K1, prevents FGF-2-mediated anti-apoptotic effects.

S6K1, Raf-1 and other PKC isoforms do not form similar complexes. RNAi-mediated downregulation of B-Raf, PKCε or S6K2 abolishes FGF-2-mediated survival. In contrast, over-expression of PKCε increases XIAP and Bcl-X_{L} levels and chemoresistance in SCLC cells. In a tetracycline-inducible system, increased S6K2 kinase activity triggers upregulation of XIAP, Bcl-X_{L} and pro-survival effects. However, increased S6K1 kinase activity has no such effect. Thus, S6K2 but not S6K1 mediates pro-survival/chemoresistance signalling.

These unforeseen results indicate divergent biological activities for S6K2 and S6K1. Thus, S6K2, unlike S6K1, is selectively recruited into a signalling complex containing PKCε and B-Raf and controls FGF-2-mediated translation of mRNA species involved in the regulation of cell death.

The present invention provides a complex comprising two or more of S6K2, PKCε and B-Raf. This complex has been found to be involved in the development of chemoresistance. Preferably the complex comprises S6K2, PKCε0 and B-Raf.

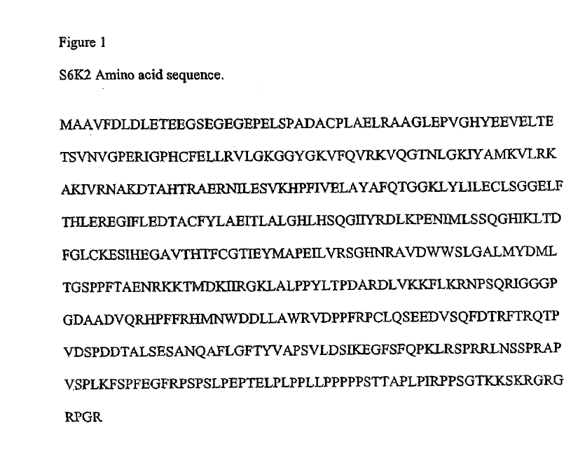

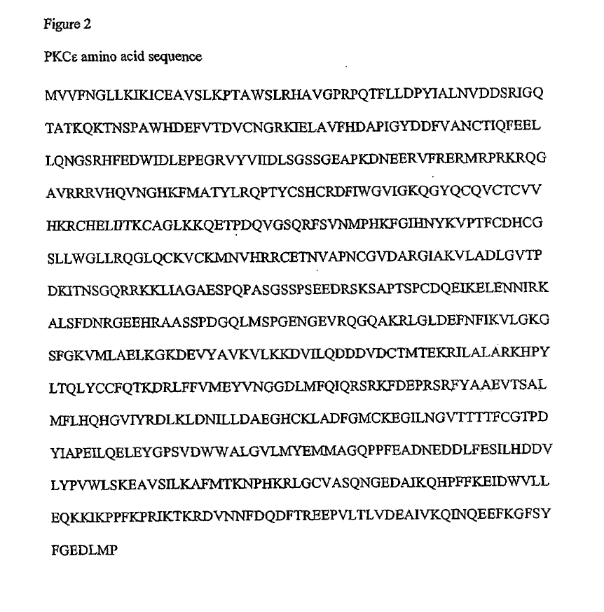

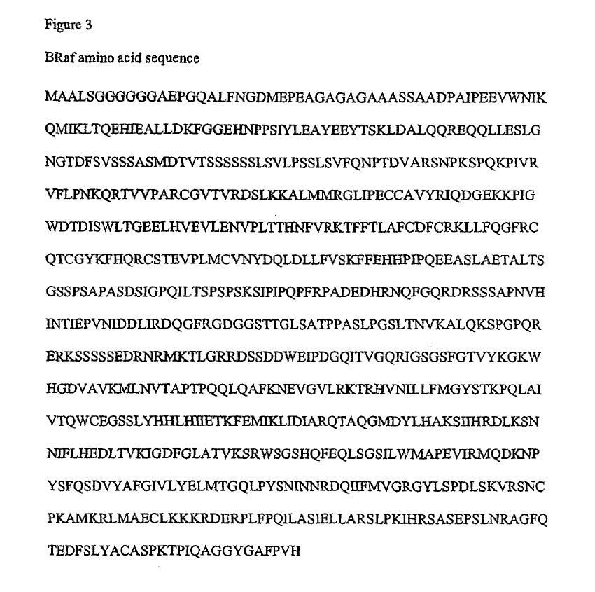

As a preferred feature of the first aspect of the complex is capable of causing chemoresistance in a cancer cell. S6K2 may comprise the sequence as set out in FIG. 1, B-Raf may comprise the sequence as set out in FIG. 2, and /or PKCε may comprise the sequence as set out in FIG. 3.

A second aspect of the invention relates to an antibody which binds specifically to the complex of the first aspect. Such an antibody may be monoclonal or polyclonal and produced and isolated by any way known in the art.

Such antibodies may be useful in the detection of the complex of the first aspect, or in the inhibition of the complex of the first aspect. The antibody may bind specifically to any of the two or more proteins S6K2, PCKε or B-Raf in the complex. The antibody may bind to an epitope that only becomes available and accessible once the proteins have formed the complex. Such an epitope can be identified by methods known in the art to the skilled person.

The term “antibody” as used herein refers to immunoglobulin molecules and immunologically active portions of immunoglobulin molecules, i.e., molecules that contain an antigen binding site that specifically binds an antigen, whether natural or partly or wholly synthetically produced. The term also covers any polypeptide or protein having a binding domain which is, or is homologous to, an antibody binding domain. These can be derived from natural sources, or they may be partly or wholly synthetically produced. Examples of antibodies are the immunoglobulin isotypes (e.g., IgG, IgE, IgM, IgD and IgA) and their isotypic subclasses; fragments which comprise an antigen binding domain such as Fab, scFv, Fv, dAb, Fd; and diabodies. Antibodies may be polyclonal or monoclonal.

It is possible to take monoclonal and other antibodies and use techniques of recombinant DNA technology to produce other antibodies or chimeric molecules which retain the specificity of the original antibody. Such techniques may involve introducing DNA encoding the immunoglobulin variable region, or the complementary determining regions (CDRs), of an antibody to the constant regions, or constant regions plus framework regions, of a different immunoglobulin. See, for instance, EP-A-184187, GB 2188638A or EP-A-239400. A hybridoma or other cell producing an antibody may be subject to genetic mutation or other changes, which may or may not alter the binding specificity of antibodies produced.

As antibodies can be modified in a number of ways, the term “antibody” should be construed as covering any specific binding member or substance having a binding domain with the required specificity. Thus, this term covers antibody fragments, derivatives, functional equivalents and homologues of antibodies, humanised antibodies, including any polypeptide comprising an immunoglobulin binding domain, whether natural or wholly or partially synthetic. Chimeric molecules comprising an immunoglobulin binding domain, or equivalent, fused to another polypeptide a re therefore included. Cloning and expression of chimeric antibodies are described in EP-A-0120694 and EP-A-0125023. A humanised antibody may be a modified antibody having the variable regions of a non-human, e.g. murine, antibody and the constant region of a human antibody. Methods for making humanised antibodies are described in, for example, U.S. Pat. No. 5,225,539.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341:544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., Science 242:423-426 (1988); Huston et al., PNAS USA 85:5879-5883 (1988)); (viii) bispecific single chain Fv dimers (PCT/US92/09965) and (ix) “diabodies”, multivalent or multispecific fragments constructed by gene fusion (WO94/13804; P. Hollinger et al., Proc. Natl. Acad. Sci. USA 90: 6444-6448 (1993)).

Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associated with each other to form an antigen binding site: antigen binding sites are formed by the association of the first domain of one polypeptide within the multimer with the second domain of another polypeptide within the multimer (WO94/13804).

Where bispecific antibodies are to be used, these may be conventional bispecific antibodies, which can be manufactured in a variety of ways (Hollinger & Winter, Current Opinion Biotechnol. 4:446-449 (1993)), e.g. prepared chemically or from hybrid hybridomas, or may be any of the bispecific antibody fragments mentioned above. It may be preferable to use scFv dimers or diabodies rather than whole antibodies. Diabodies and scFv can be constructed without an Fc region, using only variable domains, potentially reducing the effects of anti-idiotypic reaction. Other forms of bispecific antibodies include the single chain “Janusins” described in Traunecker et al., EMBO Journal 10:3655-3659 (1991).

Bispecific diabodies, as opposed to bispecific whole antibodies, may also be useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected.

A third aspect of the invention provides a method for identifying an inhibitor of the complex of the first aspect, comprising the steps of contacting a cell which expresses two or more of S6K2, PKCε and B-Raf with a test compound and determining whether a complex is formed.

Such a method may be carried out by methods known in the art. For example, the method may include a reporter gene. In this situation, if no complex is formed, the expression of a reporter gene may be switched on or off (depending on whether the complex directly or indirectly activates or inhibits expression) to indicate whether a complex has been formed. Such a reporter gene may be any known in the art that indicates clearly whether expression has been activated or inhibited, such as β-galactosidase.

A further method for identifying an inhibitor of the complex of the first aspect is also provided, comprising the steps of contacting two or more of S6K2, PKCε and B-Raf with a test compound and determining whether a complex is formed.

The method may comprise immunoprecipitation of the complex-forming proteins using an antibody that specifically binds to one of the proteins, in the presence of a test compound. If one or more of the proteins are not ‘pulled down’ by the antibody, the formation of the complex is inhibited by the compound. Of course, the skilled person appreciates that alternative ways of determining whether a test compound inhibits formation of the complex may be used, and are well known in the art.

A fifth aspect of the invention provides an inhibitor of the complex comprising two or more of S6K2, PKCε and B-Raf. Preferably, the inhibitor is identified by the method of the third and fourth aspects of the invention. The inhibitor may inhibit B-Raf expression and/or PKCΕ and/or S6K2 expression. Alternatively, the inhibitor may prevent the association of S6K2, B-Raf and/or PKCε.

A method of preventing or reversing chemoresistance in a cancer cell is also provided as a sixth aspect, comprising administering to the cell an inhibitor of the complex comprising two or more of S6K2, B-Raf and PKCε. The cancer cell to which the method applies, is preferably a small cell lung cancer (SCLC) cell. The inhibitor of the method may be RNAi, antisense RNA, ribozyme RNA or an antibody.

A seventh aspect of the invention provides a pharmaceutical composition comprising an inhibitor of the fifth aspect and a pharmaceutically acceptable adjuvant, diluent or excipient.

Pharmaceutical compositions in accordance with the invention will usually be supplied as part of a sterile, pharmaceutical composition which will normally include a pharmaceutically acceptable carrier. This pharmaceutical composition may be in any suitable form, (depending upon the desired method of administering it to a subject).

It may be provided in unit dosage form, will generally be provided in a sealed container and may be provided as part of a kit. Such a kit would normally (although not necessarily) include instructions for use. It may include a plurality of said unit dosage forms.

The pharmaceutical composition may be adapted for administration by any appropriate route, for example by the oral (including buccal or sublingual), topical (including buccal, sublingual or transdermal), or parenteral (including subcutaneous, intramuscular, intravenous or intradermal) route. Such compositions may be prepared by any method known in the art of pharmacy, for example by admixing the active ingredient with the carrier(s) or excipient(s) under sterile conditions.

Pharmaceutical compositions adapted for oral administration may be presented as discrete units such as capsules or tablets; as powders or granules; as solutions, syrups or suspensions (in aqueous or non-aqueous liquids; or as edible foams or whips; or as emulsions)

Suitable excipients for tablets or hard gelatine capsules include lactose, maize starch or derivatives thereof, stearic acid or salts thereof. Suitable excipients for use with soft gelatine capsules include for example vegetable oils, waxes, fats, semi-solid, or liquid polyols etc.

For the preparation of solutions and syrups, excipients which may be used include for example water, polyols and sugars. For the preparation of suspensions oils (e.g. vegetable oils) may be used to provide oil-in-water or water in oil suspensions. Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6):318 (1986).

Pharmaceutical compositions adapted for topical administration may be formulated as ointments, creams, suspensions, lotions, powders, solutions, pastes, gels, sprays, aerosols or oils. For infections of the eye or other external tissues, for example mouth and skin, the compositions are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical compositions adapted for topical administration to the eye include eye drops wherein the active ingredient is dissolved or suspended in a suitable carrier, especially an aqueous solvent.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents. Excipients which may be used for injectable solutions include water, alcohols, polyols, glycerine and vegetable oils, for example. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets.

The pharmaceutical compositions may contain preserving agents, solubilising agents, stabilising agents, wetting agents, emulsifiers, sweeteners, colourants, odourants, salts (substances of the present invention may themselves be provided in the form of a pharmaceutically acceptable salt), buffers, coating agents or antioxidants. They may also contain therapeutically active agents in addition to the substance of the present invention.

Dosages of the substances of the present invention can vary between wide limits, depending upon the disease or disorder to be treated, the condition of the individual to be treated, etc. and a physician will ultimately determine appropriate dosages to be used. The dosage may be repeated as often as appropriate. If side effects develop the amount and/or frequency of the dosage can be reduced, in accordance with normal clinical practice.

A method of diagnosing chemoresistance in a cancer patient is also provided as an aspect of the invention and comprises detecting of a complex comprising two or more of S6K2, B-Raf and PKCε. Detection may be by way of an antibody or any other method known to the skilled person.

Also provided is a method of diagnosing chemoresistance in a cancer patient comprising detecting the level of S6K2 activation in a cancer cell of the patient and comparing to the level of S6K2 activation in a non-cancer cell of the patient or a cell from a non-cancer patient, wherein the cancer cell is resistant to chemotherapy if the level of S6K2 activation is higher in the cancer cell than in the non-cancer cell or the cell from the non-cancer patient. Detection of the level may be by way of an antibody, quantitative immunoprecipitation and/or western blotting, or the like and comparing to a non-cancer cell or a cell from a non-cancer patient, as a reference for normal levels of S6K2 activation levels. Elevated levels of S6K2 activation indicate a likelihood of chemoresistance.

By activation, is meant S6K2 in a form capable of forming a complex and/or causing chemoresistance.

The skilled person will also appreciate that if non-elevated levels of S6K2 activation are determined in a cancer cell, i.e. similar levels to a non-cancer cell or a cell from a non-cancer patient, then the cancer cell is likely to be chemosusceptible.

A method of predicting the likelihood of a cancer cell developing chemoresistance is also provided, comprising measuring the levels of S6K2 activation in the cell at two or more time points, wherein the cancer cell is likely to develop chemoresistance if the level of S6K2 activation increases between time points.

The time points may be any reasonable interval, such as daily, weekly or monthly, depending on the type of cancer, the wellbeing of the patient, the speed of progression of the cancer and other factors. If the level of activated S6K2 does not increase between time points, then it is likely that the cancer cell will be susceptible to chemotherapy.

A method of diagnosing chemoresistance in a cancer cell comprising detecting the level of S6K2 in the cell is also provided. The level of S6K2 may be determined by any method known by one skilled in the art. The cell is likely to be chemoresistant if elevated levels of S6K2 are detected.

A twelfth aspect provides the use of an inhibitor of the complex of the first aspect in the manufacture of a medicament for the prevention or reversal of chemoresistance in a cancer cell.

All preferred features of each aspect apply to all other aspects mutandis mutatis.

The invention will now be described with reference to the following non limiting examples and figures, in which:

FIG. 1 shows the nucleic acid and amino acid sequence of S6K2

FIG. 2 shows the nucleic acid and amino acid sequence of B-Raf

FIG. 3 shows the nucleic acid and amino acid sequence of PKCε

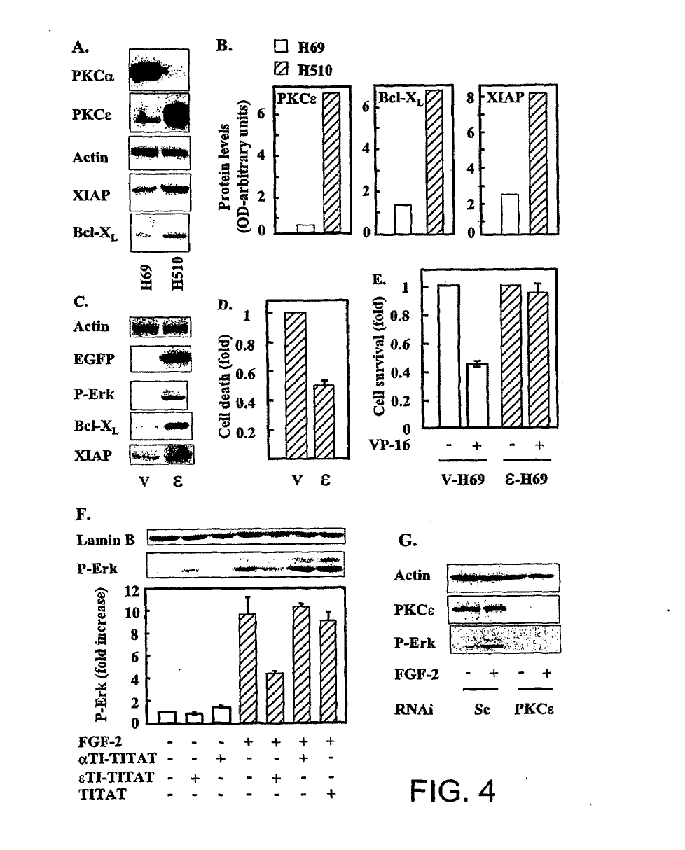

FIG. 4 shows that PKCε levels correlate with XIAP and Bcl-X_{L} expression and Erk phosphorylation in SCLC cells, wherein: (A) H69 and H510 cell lysates were Western-blotted for the expression of PKCα, PKCε, XIAP, Bcl-X_{L} and actin. (B) Representative blots from (A) were quantified by optical densitometry normalised for actin. (C) H69 cells transfected with empty (V) or a wt-PKCε-GFP expressing vector (a) were analysed for phospho-ERK, XIAP and Bcl-X_{L} levels. (D) Baseline level cell death in V-H69 and ε-H69 cells growing in 10% FCS was determined by flow cytometry using Annexin V staining. (E) ε-H69 and V-H69 cells in SFM were treated with or without 0.1 μM etoposide (VP-16) and cell numbers determined 96 h later. Conditions were performed in quadruplicates and the average cell number±SEM represented as fold over untreated. (F) H510 cells in SFM were treated with or without 40 μM ε-TITAT, ΕTI-TITAT or TITAT for 4 h prior to stimulation for 5 min with or without FGF-2 (0.1 ng/ml). Cell lysates were Western-blotted for biphospho-ERK. (F-lower panel) Results from three independent experiments were analysed by optical densitometry and represented as average±SEM fold increase over control. (G) H510 cells transfected with PKCε or scrambled (sc) siRNA were stimulated with or without FGF-2. Lysates were analysed for PKCε levels and Erk phosphorylation. (A, C, F and G) Lamin B and actin immunodetection were used as loading controls.

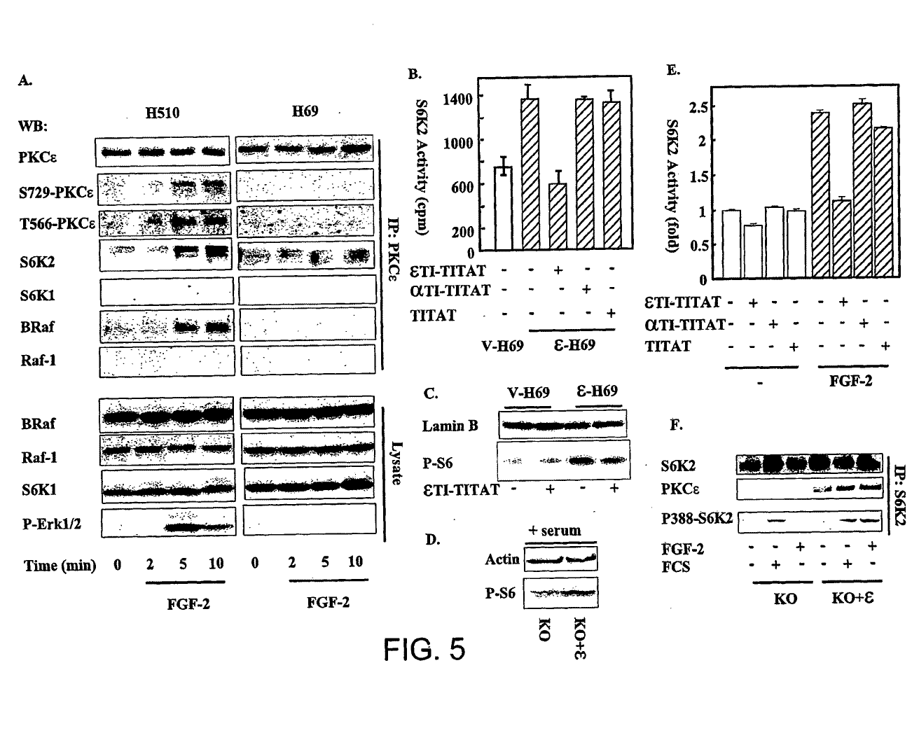

FIG. 5 shows that PKCε forms a multiprotein complex with B-Raf and S6K2 in H510 cells following FGF-2 and regulates S6K2 activity, where: (A) H510 and H69 cells in SFM were treated with FGF-2 for the times indicated. Cell lysates were subjected to immunoprecipitation with a PKCε antibody prior to Western blotting (WB) for the molecules indicated. (A-lower panel) Total cell lysate was Western-blotted as indicated. (B and E) S6K2 was immunoprecipitated from V-H69 and ε-H69

(B) or H510 cells (E) following 4 h treatment with or without εTI-TITAT, αTI-TITAT and TITAT. Immunoprecipitates were subjected to in-vitro kinase assays with S6-peptide as a substrate and the results shown are average cpm±SEM from triplicates of a representative experiment. (C) The phosphorylation of the endogenous

S6 protein from V-H69 and ε-H69 cells in SFM treated with or without εTI-TITAT was determined using a phospho-S6 antibody. (D and F) PKCε KO MEFs re-expressing (KO+ε) or not (KO) PKCα were (D) grown in 10% FCS and analysed for phospho-S6 levels or (F) stimulated with or without FGF-2 and FCS prior to S6K2 immunoprecipitation and Western blotted as indicated. (C and D) Lamin B and actin immunodetection were used as a loading control. (A-F) Results shown are representative of at least 3 independent experiments.

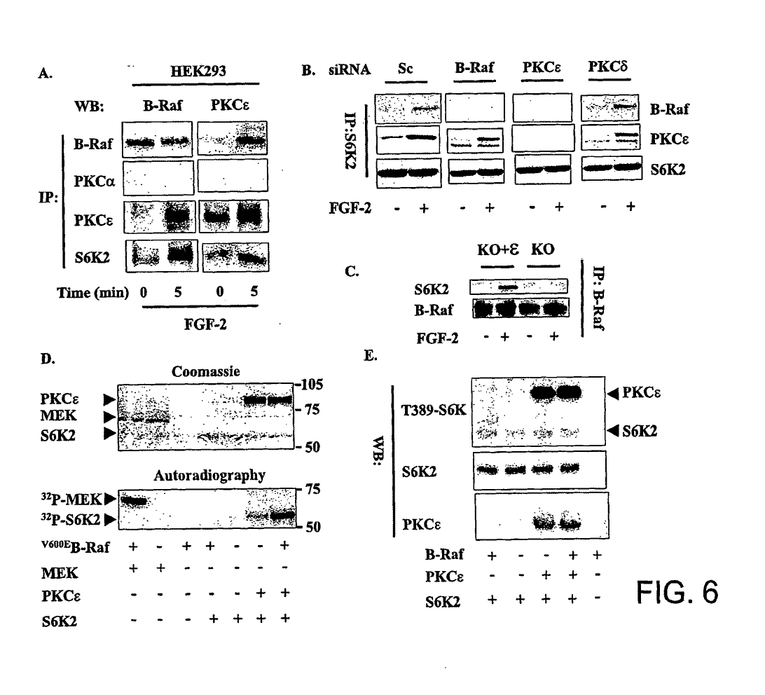

FIG. 6 shows that PKCε is required for B-Raf association with S6K2. (A) HEK 293 cells were stimulated with FGF-2 and immunoprecipitates (IP) for the molecules indicated analysed by Western-blotting (WB) for either B-Raf or PKCε (B) HEK 293 cells transfected with siRNAi for B-Raf, PKCα, PKCε, PKCδ or scramble control (sc) were stimulated with FGF-2 and S6K2 immunoprecipitates analysed by WB for B-Raf and PKCε (C) MEFs from PKCε KO mice, re-expressing (KO+ε) or not (KO) PKCε were stimulated with or without FGF-2. B-Raf immunoprecipitates were analysed by WB for S6K2. (D and E) Recombinant PKCε, ^{V600E}B-Raf and S6K2 proteins were combined as indicated and subjected to in vitro kinase assay with ³²P-γATP (D) or cold ATP (E). Recombinant GST-MEK was used as a positive control for ^{V600E}B-Raf activity. Samples were run on SDS-PAGE, Coomassie-stained (D-upper panel) or transferred to nitrocellulose (E), then exposed to an X-Ray film (D-lower panel) or subjected to WB for the molecules indicated (E). Results shown are representative of a minimum of three independent experiments.

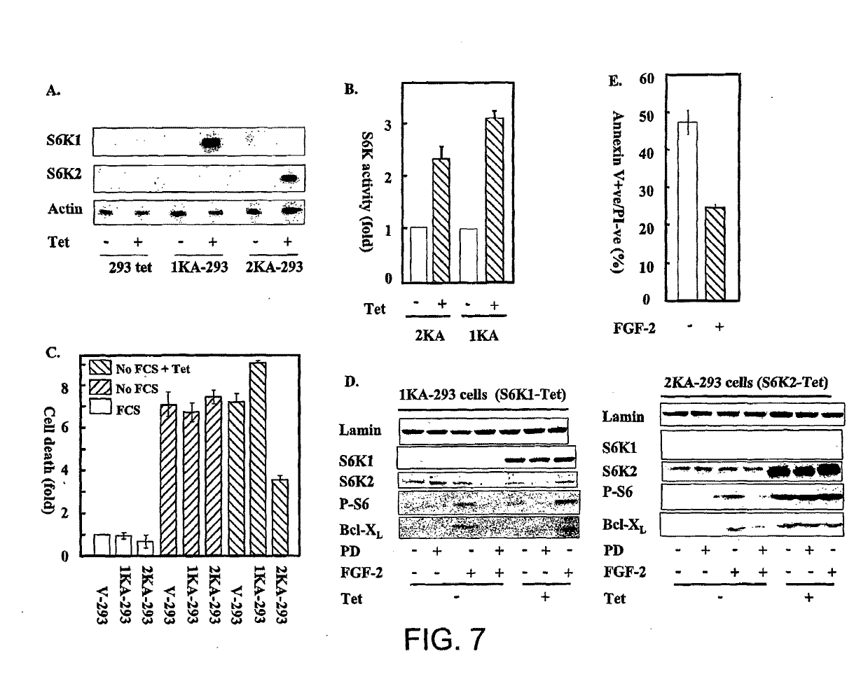

FIG. 7 shows that specific induction of S6K2 kinase activity in HEK 293 cells increase cell viability and upregulates Bcl-X_{L}. HEK 293-Tet clones transfected with an inducible vector for kinase active S6K1 (1KA), S6K2 (2KA) or empty vector were treated with tetracycline for 6 h prior to (A) Western-blotting (WB) as indicated or (B) S6K1 or 2 kinase assay using an S6 peptide as substrate. (C) V-, 1KA- and 2KA-293 cells were incubated in the absence of tetracycline with (white bars) or without (hatched bars) serum for 18 h or with tetracycline in the absence of serum (black bars) and the proportion of apoptotic cells determined using annexin V staining. (D) Cell lysates from 2KA and 1KA-293 cells treated with or without tetracycline, FGF-2 and PD098059 were analysed for Bcl-X_{L} expression and S6 phosphorylation. (E) HEK 293 cells incubated with or without FGF-2 (4h) prior to serum deprivation (18 h) were analysed by annexin V staining. (A, D, F) Actin and lamin B immunodetections were used as a loading control. (B, C, E) Results represent the average of triplicates±SEM. (A-E) Results are representative of at least three independent experiments.

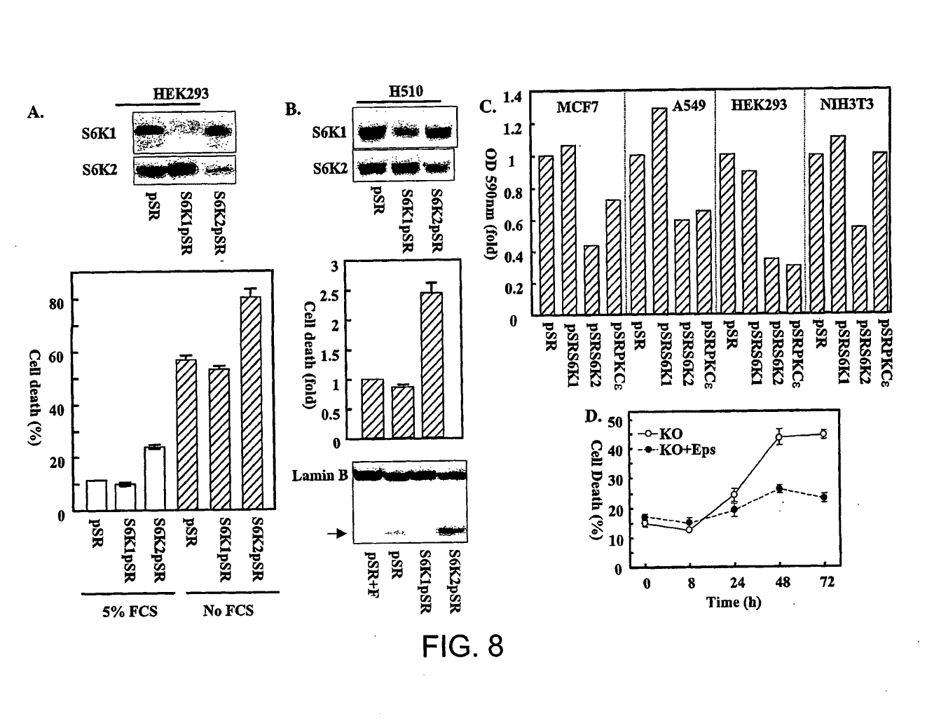

FIG. 8 shows that S6K2 and PKCε downregulation decreases cell viability and clonogenic cell growth in mammalian cells. (A) HEK 293 cells were transfected with empty-vector (pSR), or pSR encoding for S6K1 (S6K1pSR) or S6K2 (S6K2pSR) RNAi sequences. Cells were grown in 5% FCS or serum-free medium for 18 h and cell viability determined by trypan blue exclusion. (B) Lysates from H510 cells expressing pSR, S6K1pSR or S6K2pSR were Western-blotted as indicated (upper panel). The baseline cell death in 10% FCS was determined by Annexin V staining (middle panel). Lamin B cleavage was used as readout for caspase 3 activity (lower panel). pSR cells treated with FGF-2 (pSR+F) were used as negative control (C) MCF-7, A549, HEK 293 and NIH3T3 cells were transfected with the indicated pSR shRNAi constructs and grown in 5% FCS for 10 days. The OD of crystal violet stained colonies was determined at 590 nm. For each cell line, results were normalised for absorbance found in pSR empty-vector cells. (D) KO or KO+ε MEFs were plated in the absence of FCS for the times indicated and the proportion of Trypan blue-positive cells determined. For (A-lower panel) and (B-middle panel) results represent the average of triplicates±SEM. For (A-C), the results shown are representative of at least three independent experiments.

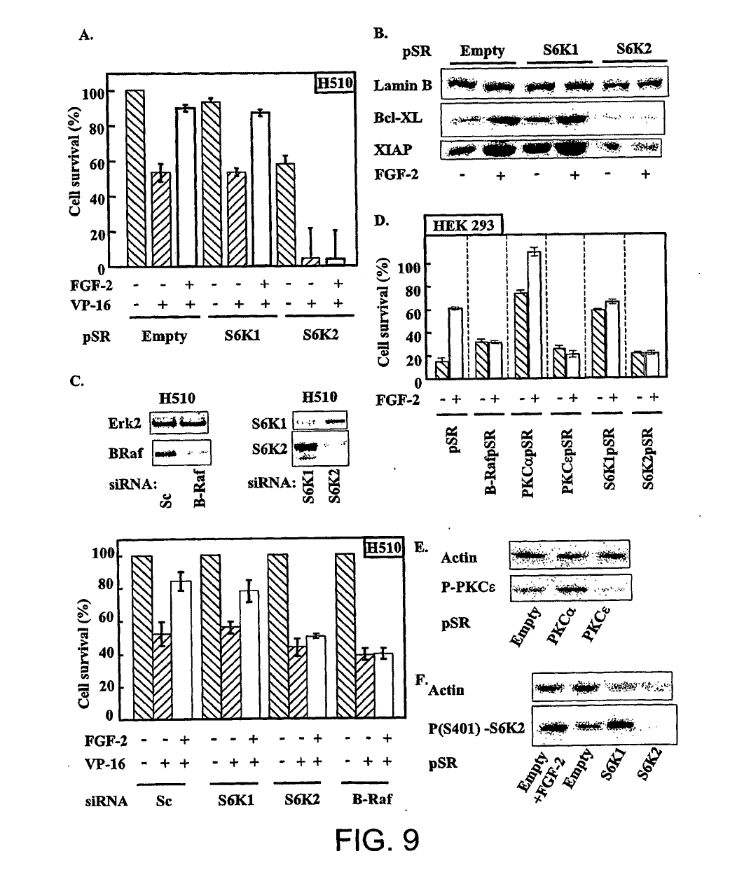

FIG. 9 shows that S6K2, but not S6K1, downregulation prevents FGF-2-mediated survival of H510 and HEK 293 cells. (A-C) H510 cells were subjected to downregulation of the indicated proteins either by pSR RNAi retroviral vectors (A,B) or oligonucleotide RNAi (C). (A) Cells were preincubated with or without FGF-2 prior to etoposide treatment (VP-16). (B) Lysate from H510 cells infected with the indicated vectors and treated as shown were Western-blotted for XIAP and Bcl-X_{L}. (C) H510 cells treated with oligonucleotide RNA is were (Upper Panel) lysed and Western-blotted as indicated or (Lower Panel) treated as described in (A). (D-F) HEK 293 cells were subjected to downregulation of the indicated proteins by transfection of RNAi-encoding pSR vectors. (D) Cells were pre-incubated with or without FGF-2 prior to serum depletion. (A and D) Survival was determined by trypan blue exclusion. (E) HEK 293 cells transfected as indicated were Western-blotted for phosphoS729-PKCε (P-PKCε). (F) Transfected HEK 293 cells incubated with or without FGF-2 were Western blotted for phosphoS412-S6K2. (A,C,D) Results are averages±SEM of quadruplicates and (A, C) normalised to pSR (A) or Sc (C). (B, E, F) LaminB or actin immunodetection were used as loading control. (A-F) Results are representative of at least three independent experiments.

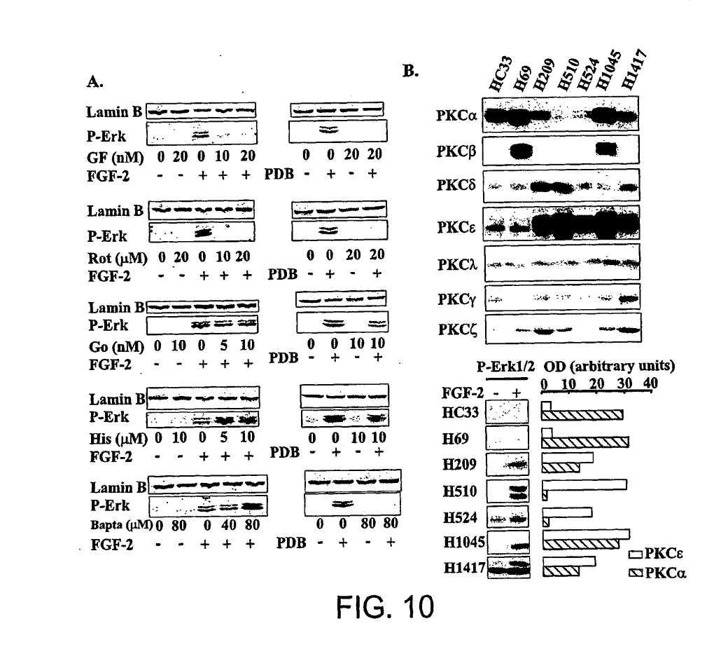

FIG. 10 shows that (A, B, C and D) PKCε controls FGF-2-mediated Erk phosphorylation in SCLC cells. (A) H510 cells were treated with or without a dose range of GF109203X (GF), Gö6976 (Go), Hispidin (His), BAPTA (BA) or Rottlerin (Rot) for 1 h prior to stimulation for 5 min in the presence or absence of either FGF-2 (0.1 ng/ml) or PDBu (400 nM). Cell lysates were analysed by SDS-PAGE/Western-blotting for biphospho-ERK. Lamin immunodetection was used as loading control. (B-top panel) Equal protein amounts from SCLC cell lines were compared for their PKC expression pattern. (B-bottom panel) SCLC cells in SFM were stimulated with and without FGF-2 for 5 min and cell lysates analysed by SDS-PAGE/WB for Erk phosphorylation. PKCε and PKCα levels (ODs from left panel) were compared to the ability to phosphorylate Erk in these SCLC cell lines. Results shown are representative of at least three independent experiments.

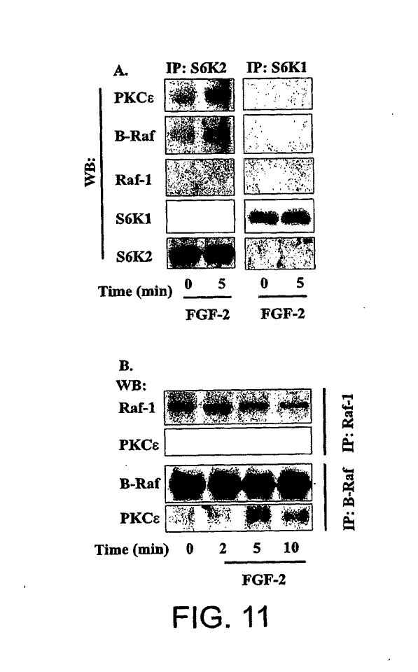

FIG. 11 shows that: PKCε forms a multiprotein complex with B-Raf and S6K2 in H510 cells. (A and B) H510 cells in SFM were treated with FGF-2 for the times indicated. Cell lysates were subjected to immunoprecipitation with either S6K1 or 2

(A), B-Raf or Raf-1 (B) antibodies prior to SDS-PAGE/Western Blotting (WB) for S6K1 and 2, B-Raf, Raf-1 and PKCε. (A and B) Results shown are representative of at least three independent experiments.

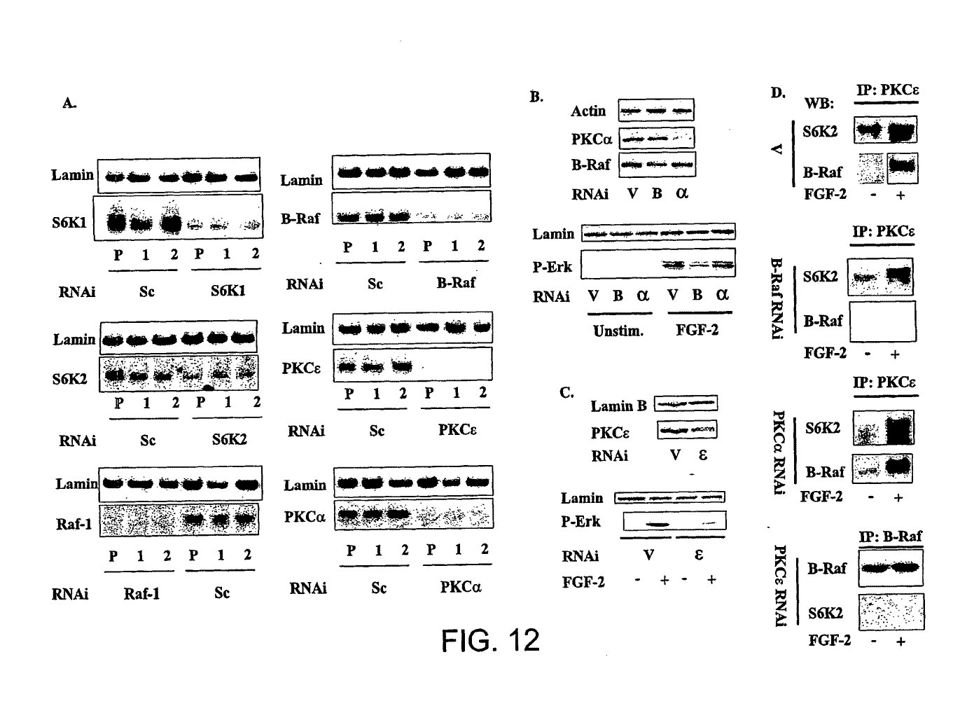

FIG. 12 shows that: PKCε is required for B-Raf association with S6K2. (A) Efficacy of single siRNAi oligonucleotide sequences (1 and 2) and Smartpools (P) directed against S6K1, S6K2, PKCe, PKCa, B-Raf and Raf-1. HEK293 cells were transfected with the oligonucleotides and lysates analysed for target downregulation 48 h later by SDS-PAGE/WB. (B, C and D) PKCα(a) and B-Raf (B) or PKCε (ε) were downregulated using pSR retroviral RNAi vectors in HEK293 cells and compared to empty vector only (V) transfected cells for their expression of the RNAi targets and their ability to phosphorylate Erk (B and C) or form the S6K2/PKCε/B-Raf multiprotein complex (D) in response to FGF-2. (A-D) Results shown are representative of at least three independent experiments.

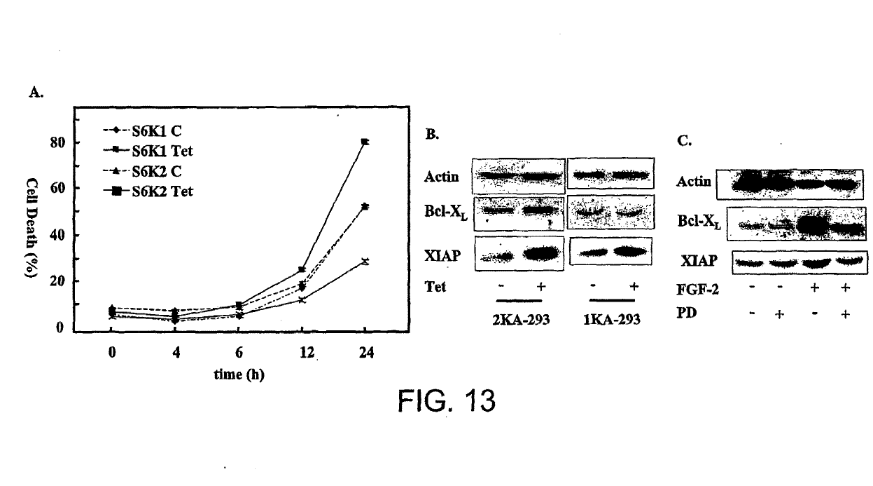

FIG. 13 shows that: S6K2 kinase activity protects HEK293 cells from serum deprivation and induces expression of Bcl-X_{L} and XIAP. (A and B) HEK293 expressing tetracycline-inducible kinase-active S6K1 (1KA) or 2 (2KA) were treated with or without tetracycline for 6 h (B) or the time indicated (A). (A) Cells were grown in the absence of FCS and a cell death time-course performed. Cell death was assessed microscopically by determining Trypan blue positivity. Results shown are averages±SEM of triplicates. (C) HEK293 cells were treated for 1 h with or without 25 μM PD098059 prior to stimulation with FGF-2 for 4 h. (B and C) Cell lysates were analysed by SDS-PAGE/WB for the levels of Bcl-X_{L} and XIAP. Actin was used as a loading control. Results shown are representative of at least three independent experiments.

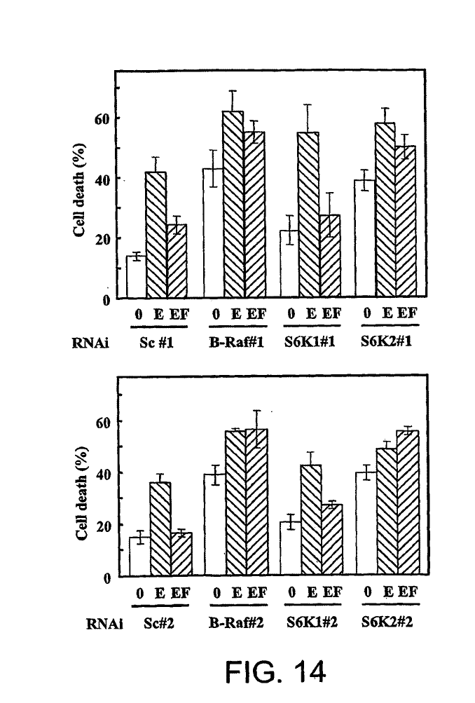

FIG. 14: S6K2 and B-Raf but not S6K1 single siRNA sequences prevent FGF-2-mediated rescue of etoposide treated H510 cells. H510 cells grown in SFM were transfected with either of two siRNA single sequences (#1 and #2 as shown in FIG. 3A) targeting S6K1, S6K2 or B-Raf as indicated. Two non-targeting sequences (sc#1 and 2) were used as controls. Cells were pre-incubated for 4 h with FGF-2 (F) prior to etoposide (E) treatment. Cell death was determined microscopically by Trypan blue exclusion. Results shown are average±SEM of triplicates and are representative of at least three independent experiments.

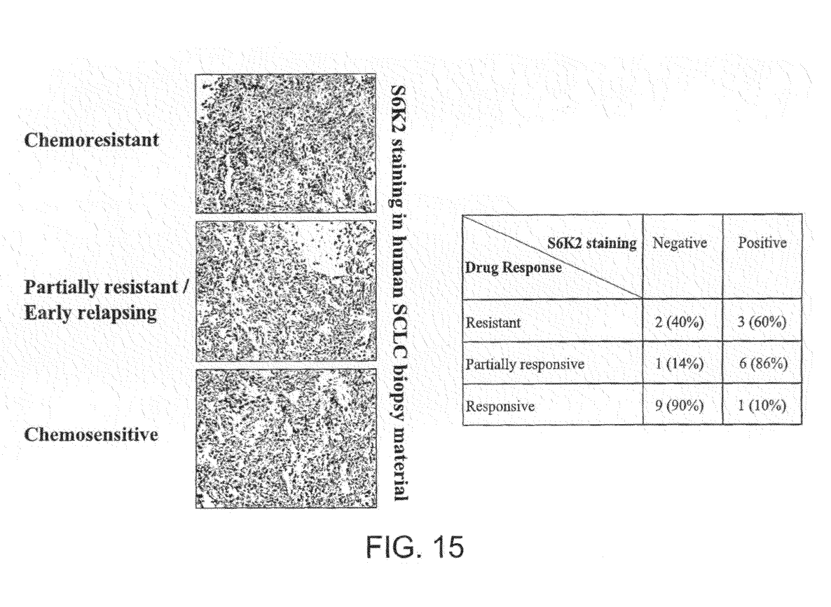

FIG. 15 shows that: S6K2 staining correlates with chemoresistance in human SCLC biopsy material. Formalin fixed and paraffin embedded biopsies from 22 patients with SCLC and NSCLC at presentation were sectioned and immunostained using a mouse anti-S6K2 monoclonal antibody (provided by Prof Gout, UCL, London) and Envision detection system (DAKO). Specificity for the target protein was controlled for by using standard protocols including known positive (H510) and negative (Type II pneumocytes) samples, irrelevant antibody and competing S6K2. The pathologist (Dr Neil Sebire, Hammersmith Hospitals) was blinded to the clinical outcome data to avoid reporting bias. The study and on going collection of SCLC and NSCLC biopsy material has been reviewed and approved by our local ethics review board.

Upper Panel: strong S6K2 immunostaining seen in most cancer cells in a biopsy from a patient with chemoresistant tumour (original magnification×100).

Middle Panel: focal areas of moderate S6K2 staining in a biopsy from a patient with partially chemoresistant disease (original magnification×100).

Lower Panel: absence of S6K2 staining in a biopsy from a patient with chemosensitive disease (original magnification×100).

These results were seen in 2 of 4 chemoresistant patients, 3/3 partially chemoresistant and 6/6 chemosensitive patients with SCLC. To substantiate these results, we also examined S6K2 staining levels in biopsies from several NSCLC patients. In one patient who was resistant to therapy the tumour was diffusely positive, three of four early relapsing patients, the tumours were focally positive for S6K2 staining whilst three of four chemosensitive tumours were negative. The combined results of staining in both SCLC and NSCLC biopsies are summarised in the adjoining table. Together, these results suggest that S6K2 protein expression levels in biopsies from patients with lung cancer correlates with chemoresistance.

### EXAMPLES

### Materials and Methods

Cell Culture

SCLC cell lines were maintained as previously described (Pardo et al., 2001). For experimental purposes, cells were grown in SFM (RPMI 1640 supplemented with 5 μg/ml insulin, 10 μg/m1 transferrin, 30 nM sodium selenite, 0.25% bovine serum albumin) and used after 3 to 7 days. A549, HEK293, HEK293Tet, NIH-3T3, MCF-7 and Cos 7 cells were grown in DMEM medium containing 10% FCS at 37° C., 10% CO₂. For experimental purpose, HEK 293 cells were placed in serum-free DMEM for 6 h prior to growth factor stimulation.

Establishment of Transgene-Expressing Cell Lines

H69 cells were transfected with pEGFP constructs encoding wild type PKCε using Lipofectin as per the manufacturer instructions and cells were selected in 1 mg/m1 G418. RNAi-expressing H510 cells were established by infecting H510 cells with an amphotropic virus coding for the murine ecotropic receptor (EcoR). Following selection with G418, cells were infected using murine retroviruses encoding for PKCαPKCε, S6K1, S6K2, B-Raf or Raf-1 short-hairpin RNAi. Stable gene downregulation was achieved by culturing the cells in the presence of 2 μg/m1 puromycin. Transient expression of B-Raf, Raf-1, PKCε, S6K1 or S6K2 RNAi was achieved by transfecting A549, HEK293, NIH-3T3, MCF-7 and Cos 7 cells with the relevant pSR construct using Lipofectamin Plus. Transgene expression or downregulation of target proteins were assessed by Western blotting.

Establishment of Tetracycline-Inducible S6K1 and S6K2 Cell Lines

HEK293Tet-on cells (Invitrogen) at 70% confluency were transfected with 25 μg of pCDNA4-S6K2-T412D, pCDNA4-S6K1-T401D or pCDNA4 (control) using calcium phosphate precipitation. Cells were selected in 50 mg/ml zeocin. 15 colonies from each transfection were isolated using cylinders, and clonal cell lines were established and tested for expression of S6K1 or S6K2 upon incubation with 1 mg/ml of tetracycline by western blot analysis.

Cell Death Assay

SCLC cells (5×10⁴ cells/ml SFM) were pre-treated with or without 0.1 ng/ml FGF-2 for 4 h prior to treatment with 0.1 μM etoposide and incubated at 37° C. for 96 h. HEK 293-Tet cells were plated in 48-well plates (10⁴ cells/well), pre-treated with or without 0.1 ng/ml FGF-2 for 4 h and cell death induced by serum removal for 18 h. The proportion of cell death was either determined by trypan blue exclusion or by Annexin V staining and flow cytometry as previously described (Pardo et al., 2002).

Cell-Permeable PKCε and PKCα Translocation Inhibitor Peptide

The PKCε translocation inhibitor (EAVSLKPT) and PKCα translocation inhibitor (SLNPEWNET) (Souroujon and Mochly-Rosen, 1998; Yedovitzky et al., 1997) were made cell-permeable by linkage to the HIV-derived TITAT sequence (GRKKRRQRRRPPQ). H510 cells in RPMI were incubated for 4 h with 40 μM of either translocation inhibitor peptides or TITAT prior to further treatments. The activity of these inhibitors on ERK phosphorylation was assessed by Western Blotting.

Co-Immunoprecipitation Experiments

SCLC cells grown in SFM were washed in RPMI 1640, and 2×10⁶ cell aliquots were incubated in this medium for 30 min at 37° C. HEK 293 cells were washed and incubated in DMEM for 6 h. Cells were then stimulated using FGF-2 for the time shown in the figure legends. Cells were lysed at 4° C. in 1 ml of lysis buffer, lysates clarified by centrifugation at 15,000 g for 10 min and immunoprecipitation performed for 1.5 h using the relevant antibody together with either Protein A or G.

S6K1/2 Immune Complex and In Vitro Kinase Assays

See supplementary information

Clonogenic Growth Assays

A549, HEK293, NIH-3T3, MCF-7 and Cos 7 cells transfected with the relevant construct were plated in 6-well plates (2×10³ cells/plate) and left to grow for 10 days 37° C./10% CO₂ in DMEM/5% FCS. Cells were then stained with crystal violet, colonies solubilised using a 10% acetic acid solution, and absorbance measured at 595 nm.

RNAi Sequences

RNAi-mediated downregulation of PKCα, PKCε, S6K1, S6K2, B-Raf and Raf-1 was achieved using short-hairpin sequences cloned into pSUPER Retro constructs or oligonucleotide siRNA. See supplementary information for sequences.

Oligonucleotide Nucleofection

1.5×10⁶ SCLC cells grown in serum medium were transfected following the manufacturer's instructions with 6 μl of 20 μM siRNA in 100 μl of Nucleofector Solution V using the program T-16 on the Amaxa Nucleofector. Following transfection, cells were transferred into RPMI/10% FCS overnight before they were used for analysis.

Immune Complex Kinase Assays

V-H69 and -H69 cells in SFM were washed three times in RPMI 1640, and 2×106 cell aliquots were incubated in this medium for 30 min at 37° C. Cells were treated in the presence or absence of 40 μM (TI-TITAT, (TI-TITAT or TITAT peptide for 4 h as indicated, prior to performing an immune complex kinase assay for S6K1 or S6K2 as described (Pardo et al., 2001). In separate experiments, HEK293Tet cells expressing tetracycline-inducible kinase active cytoplasmic forms of S6K1 or S6K2 were incubated with or without tetracycline prior to lysis and immune complex kinase assay.

In Vitro Kinase Assay

0.5 μg recombinant His-S6K and 4 μg recombinant PKCε were incubated on ice in 50 mM TRIS (pH 7.5), 100 mM NaCl, 0.1 mM EDTA and 0.1% TritonX-100, 0.3 (v/v)% β-mercatptoethanol and 1 mM Na3VO4 in the presence or absence of recombinant active V600EB-Raf. The reaction was started by adding an ATP-mix resulting in a final concentration of 100 μM, ATP, 10 mM MgCl2 with or without 33 nCi/μL [γ-³²P]ATP and incubated for 30 min at 30° C. As a positive control recombinant GST-MEK was used as a substrate for V600EB-Raf. Reactions were terminated in SDS-sample buffer and analysed by SDS-PAGE and autoradiography.

RNAi Sequences

RNAi-mediated downregulation of PKCα, PKCε, S6K1, S6K2, B-Raf and Raf-1 was achieved using short-hairpin sequences cloned into pSUPER Retro constructs or oligonucleotide siRNA and are listed in the supplementary data. For pSUPER Retro-mediated downregulation, each protein was simultaneously targeted using three different short-hairpin sequences. Sequences were as follow: PKCα, CAAGGCTTCCAGTGCCAAG, GGAACACATGATGGATGGA, CATGGAACTCAGGCAGAAA; PKCε, TCTGCGAGGCCGTGAGCTT, CTACAAGGTCCCTACCTTC, GGAAGGGATTCTGAATGGT; S6K1, GGTTCTGGGCCAGGGATCC, GCTCTATCTCATTCTGGAC, CATCATCACTCTGAAAGAT; S6K2, GGGGGGCTATGGCAAGGTG, CGGAATCCCAGCCAGCGGA, GATACGGCCTGCTTCTACC; B-Raf, CAACAGTTATTGGAATCTC, CCTATCGTTAGAGTCTTCC, GAATTGGATCTGGATCATT; Raf-1, CAGTGGTCAATGTGCGAAA, GAACTTCAAGTAGATTTCC, CATCAGACAACTCTTATTG. Oligonucleotide siRNA against S6K2 and S6K1 were purchased from Dharmacon as SMARTpools. Sequences were as follow: S6K2, GCAAGGAGUCUAUCCAUGAUU, GACGUGAGCCAGUUUGAUAUU, GGAAGAAAACCAUGGAUAAUU, GGAACAUUCUAGAGUCAGUUU; AS, 5′-PACUGACUCUAGAAUGUUCCUU; S6K1, GCAGGAGUGUUUGACAUAG, GACAAAAUCCUCAAAUGUA, CAUGGAACAUUGUGAGAAA, CCAAGGUCAUGUGAAACUA. Oligonucleotide targeting of B-Raf was achieved using a single sequence: AAAGAAUUGGAUCUGGAUCAU.

Reagents

Etoposide was purchased from Calbiochem. PKCα, PKCβ, PKδ, PKCλ, PKCγ, PKCζ, PKCα, PKCε, Bcl-X_{L} and XIAP antibodies were purchased from Becton Dickinson. The phospho-PKCαand phosphor-S6 protein antibody was from Cell Signalling. The phospho-PKCε antibody against Ser729 and an additional PKCε antibody (for Western-blotting only) were obtained from Upstate. The phospho-PKCε antibody against Thr566 was as described previously (Parekh et al., 1999). S6K1, B-Raf, Raf-1, Lamin B and Actin antibodies were purchased from Santa Cruz. The S6K2 antibody was as described previously (Gout et al., 1998). Protein A and G were obtained from Amersham. Lipofectin, Lipofectamin Plus, G418, zeocin and puromycin were obtained from Invitrogen. The activated ERK antibody, etoposide, polybrene and crystal violet were obtained from Sigma. FGF-2, PD098059, Gö6976, Hispidin, BAPTA, Rottlerin and GF109203X were purchased from Calbiochem.

### Example 1

### PKCε Levels Correlate With Bcl-X_{L} and XIAP Expression and Cell Survival

It was initially investigated whether PKCε levels correlated with the expression of Bcl-X_{L} and XIAP, known regulators of H510 and H69 SCLC cell survival (Pardo et al., 2002; Pardo et al., 2003). Western blots revealed that H69 cells with low levels of PKCε displayed lower Bcl-X_{L} and XIAP expression than H510 cells that contained high levels of PKCe (Mg. 4A and 4B). A similar correlation between PKCε XIAP and Bcl-X_{L} levels existed in seven additional SCLC cell lines but was not seen for other PKCs, including PKCδ (data not shown). Also, in most cell lines, an inverse correlation between the levels of PKCα and PKCε seemed to exist (FIG. 4A and FIG. 10C]). These results suggested that PKCε might control the expression of Bcl-X_{L} and XIAP in SCLC cells. Indeed, c-H69 cells overexpressing wild-type PKCε showed increased levels of both Bcl-X_{L} and XIAP compared to vector-alone (V-H69) cells (FIG. 4C). The ε-H69 cells also showed increased background phosphorylation of ERK (FIG. 4C), enhanced survival in normal culture conditions (FIG. 4D) and resistance to etoposide (VP-16) induced cell death (FIG. 4E).

### Example 2

### FGF-2-Induced ERK Phosphorylation is Mediated by PKCε

FGF-2-induced MEK/ERK signalling increases Bcl-2, Bcl-X_{L}, XIAP and cIAP1 expression in SCLC cells (Pardo et al., 2002; Pardo et al., 2003). It was investigated whether PKCs such as PKCε might mediate FGF-2-induced MEK/ERK signalling in H510 cells. To investigate this notion the effect of the cell permeable Ca²⁺ chelator BAPTA was tested and a panel of inhibitors including Gö6976, Hispidin, Rottlerin and GF109203X which target PKCα/β1, PKCβ, nPKCs or is non-selective, respectively. Only GF109203X and Rottlerin inhibited FGF-2-mediated ERK phosphorylation in H510 cells although the compounds were all active as they blocked acute PDBu-induced ERIC phosphorylation (FIG. 10A). Rottlerin inhibits both PKCδ (Gschwendt et al., 1994) and PKCε (Davies et al., 2000), but taken together with previous findings, it appears that PKCE is the critical mediator of FGF-2-induced ERK signalling in 11510 cells. In agreement with this, comparison of PKC isoform expression levels in 7 SCLC cell lines showed that only PKCε correlated with FGF-2-induced ERK phosphorylation (FIG. 10B)

To confirm the involvement of PKCε in FGF-2-mediated ERK signalling, a cell permeable translocation inhibitor peptide for PKCε (εTI-TITAT) was used and compared with a PKCα inhibitor (αTI-TITAT) or carrier peptide alone (TITAT) (Vives et al., 1997). Treatment with εTI-TITAT led to a 60% inhibition of ERK phosphorylation in response to FGF-2 (FIG. 4F). In contrast, neither αTI-TITAT nor TITAT inhibited this response. To verify these findings, PKCs were down-regulated in H510 cells using either synthetic short interfering RNA (siRNA) as smart pools (P) or deconvoluted individual siRNA's. Preliminary experiments confirmed the efficacy and selectivity of these pooled or individual siRNA's (FIG. 12A and data not shown). FIG. 4G demonstrates that such downregulation completely prevented FGF-2-induced ERK activation while scrambled siRNA had no effect. Similar results were seen in HEK293 cells using either the same siRNA molecules (data not shown) or pSR vectors encoding short-hairpin RNAi (shRNAi) targeting distinct sequences within PKCε (FIG. 12C). In contrast, parallel experiments targeting other PKC isoforms including PKCδ had no such effect (data not shown). Taken together, these results implicate PKCε in FGF-2-mediated ERK signalling in both H510 and HEK293 cells.

### Example 3

### PKCε, B-Raf and S6K2 Form a Multiprotein Complex Following FGF-2 Treatment in H510 Cells

MEK/ERK signalling is required for S6K2 activation by FGF-2 in H510 SCLC cells. However, in H69 cells, where the FGF receptors are uncoupled from MEK/ERK,

FGF-2 fails to activate S6K2 (Pardo et al., 2001) and also fails to induce chemoresistance (Pardo et al., 2002). Hence further investigation of these two cell lines provided a valuable opportunity to elucidate the molecular mechanisms by which PKCs integrates signals to both S6K2 and ERK following FGF-2 stimulation. Lysates from H510 and H69 cells treated with or without FGF-2 were co-immunoprecipitated to identify potential differences in PKCε phosphorylation and binding partners. Since phosphorylation of T566 and 5729 on PKCE are known to correlate with activity of this kinase (Cenni et al., 2002), phospho-specific antibodies to these sites were employed in the analysis. In H510 cells, FGF-2 increased phosphorylation of both these sites within 5 min (FIG. 5A upper panel), a time-course consistent with ERK phosphorylation (FIG. 5A lower panel). This correlated with the co-immunoprecipitation of S6K2 and B-Raf but not S6K1 or Raf-1. In contrast, in H69 cells, FGF-2 failed to induce phosphorylation of residues T566 or S729 on PKCε (FIG. 5A). Moreover, FGF-2 did not trigger co-association of S6K2, S6K1, B-Raf or Raf-1 with PKCe and, as previously described, failed to induce ERK phosphorylation in these cells. However, these proteins were easily detected in total cell lysates from H69 cells (FIG. 5A-lower panel). Thus, FGF-2 appears to activate PKCe and may induce the formation of a novel signalling complex comprising PKCε/B-Raf and S6K2 in H510 cells.

The protein identities of this new FGF-2-induced signalling complex were confirmed in H510 cells, by repeating the co-immunoprecipitation experiments using antibodies directed against S6K2, S6K1, Raf-1 or B-Raf (FIGS. 11A and B). As B-Raf activation has repeatedly been implicated in ERK signalling (Calipel et al., 2003; Dillon et al., 2003; Erhardt et al., 1999; Peraldi et al., 1995; Wan et al., 2004), the results suggest the existence of a signalling module in which both B-Raf and PKCε might be required for ERK phosphorylation downstream of FGF-2. In addition, the association of S6K2 to PKCα in an FGF-2-dependent manner raised the possibility of this PKC isoform being involved in S6K2 activation.

To investigate this, the basal S6K2 activity in the PKCe-over-expressing H69 cells (ε-H69) was compared with that in the empty-vector transfected cells (V-H69). Background S6K2 activity was increased by 2-fold in ε-H69 as compared to V-H69 cells (FIG. 2B). This increase was dependent on PKCε basal activity as PKCε inhibition with εTI-TITAT lowered S6K2 activity in ε-H69 cells to a level comparable to that found in V-H69 cells (FIG. 5B). In contrast, incubation of ε-H69 cells with the PKCα translocation inhibitor (αTI-TITAT) or TITAT alone had no effect on S6K2 activity. This elevated S6K2 activity correlated with increased S6 phosphorylation in vivo, which was prevented by εTI-TITAT (FIG. 5C). Conversely, in PKCε null MEFs (KO), re-expression of PKCε (KO+ε) (Ivaska et al., 2002), enhanced S6 phosphorylation in response to serum or FGF-2 (FIG. 5D and data not shown). The KO+ε MEF cell line shows equivalent physiological responses to wild-type MEF cell lines despite slightly increased PKCe expression levels (Ivaska et al., 2002; Kermorgant et al., 2004). Moreover, in H510 cells, FGF-2-induced S6K2 activation was also inhibited by εTI-TITAT but not by αTI-TITAT or TITAT (FIG. 5E). To further substantiate the role of PKCε in S6K2 activation by FGF-2 or serum, PKCε null MEFs (KO) were compared with the KO+ε cells for co-association of PKCε with S6K2 and phosphorylation of T388 in the C-terminal of S6K2, a site known to correlate with activation of this kinase. Only the KO+ε MEFs showed FGF-2-induced association of PKCε with S6K2, which paralleled enhanced phosphorylation of S6K2 upon T388 (FIG. 5F). Serum also induced co-association of these two kinases, but unlike FGF-2, stimulated T388 phosphorylation both in the KO and KO+ε cells. Taken together, these data demonstrate that PKCε, B-Raf and S6K2 are part of a multi-protein complex that forms in response to FGF-2 stimulation and regulates S6K2 activity.

### Example 4

### A PKCε, B-Raf and S6K2 Complex Forms in HEK293 Cells: PKCε Down-Regulation Disrupts B-Raf Association with S6K2

To determine whether FGF-2 could induce formation of the BRaf/PKCε/S6K2 complex in additional cell types, we utilized HEK293 cells, KO and KO+ε cells were utilized. B-Raf could be co-immunoprecipitated with either PKCε or S6K2 following FGF-2 stimulation in 293 cells or KO+ε but not in the KO cells lacking PKCε (FIGS. 6A, 6C 5F and data not shown). Moreover, neither PKCα, Raf-1 nor S6K1 associated with S6K2 (data not shown). Thus, induction of this novel signaling complex by FGF-2 is not restricted to SCLC cells.

To identify the possible sequence of interactions involved in the assembly of this multiprotein complex B-Raf, PKCε or as controls PKCα and PKCδ were selectively down-regulated and the effect on complex formation assessed. HEK293 cells were transfected with pooled or individual siRNA or pSR vectors encoding shRNAi. Target selectivity and ability to impair FGF-2-induced ERK phosphorylation was determined (suppl FIG. 6A-C and data not shown). The effect of down-regulating these proteins on the associations of B-Raf and PKCε with S6K2 in response to FGF-2 was assessed. Knockdown of PKCδ or α or use of a scrambled RNAi had no effect on the formation of the complex (FIG. 6B and FIG. 12D). In the absence of B-Raf, PKCε still associated with S6K2. However, B-Raf failed to associate with S6K2 in the absence of PKCε. (FIG. 6B and FIG. 12D). Importantly, identical results were seen with siRNA or shRNAi strategies targeting different sequences, although the former was more efficient at target protein knockdown. This suggests that while PKCε association to S6K2 could be direct, B-Raf association to S6K2 requires PKCε. In agreement with this, FGF-2 only induced association of B-Raf with S6K2 in the KO+ε but not KO cells (FIG. 6C).

To further investigate this and examine whether PKCε and/or B-Raf could modulate the phosphorylation status of S6K2, purified preparations of these kinases were co-incubated in various combinations with ³²Pi-ATP. When activated B-Raf (^{V600E}B-Raf) was co-incubated with S6K2 no phosphorylation of S6K2 was seen although in parallel experiments, ^{V600E}B-Raf could efficiently phosphorylated MEK (FIG. 6D lower panel). In contrast, PKCE induced a marked phosphorylation of S6K2, which was further enhanced by the addition of ^{V600E}B-Raf (FIG. 6D lower panel). Coomassie staining confirmed that these changes were not a consequence of unequal loading of the added kinases (FIG. 6D upper panel). Repetition of this experiment using cold ATP and western blotting for T388S6K2 (also detects T389S6K1 and an equivalent site on PKCε) showed that this was not the phosphorylation site on S6K2 induced by PKCε or ^{V600E}BRaf (FIG. 6E). Collectively, these results indicate that PKCε can directly associate and phosphorylate S6K2 whilst B-Raf likely requires the presence of PKCε to join the complex.

### Example 5

### S6K2, but not S6K1, Kinase Activity Increases Survival and Upregulates of Bcl-X_{L} and XIAP

Since FGF-2-induced cell survival requires PKC which forms a complex with B-Raf and S6K2, but excludes S6K1, it is plausible that the two S6K isoforms differ in their ability to control cell survival. To test this hypothesis, we generated several clones of HEK293Tet cells (Invitrogen) expressing kinase active tetracycline-inducible constructs of the cytoplasmic forms of both S6K1 and S6K2. Tetracycline selectively increased the protein levels of transfected S6K isoforms with no effect on the parental cell line (293Tet) in all clones tested (FIG. 7A and data not shown). In vitro kinase assay and western blotting for S6 phosphorylation confirmed that tetracycline treatment increased the activity of the corresponding kinase (FIGS. 7B, D) similar to that seen following FGF-2 stimulation ((Pardo et al., 2001) and data not shown). The effect of this selective increase in kinase activity on the ability of HEK293Tet cell clones to survive serum starvation was then assessed. In the absence of tetracycline the proportion of cell death was similar in the KA-S6K1, KA-S6K2 and vector alone HEK293Tet cells. Following tetracycline exposure only the KA-S6K2 over-expressing cells had reduced cell death (FIG. 7C), seen between 12-24 h after serum withdrawal (FIG. 13A). These results could not be attributed to enhanced cell proliferation as KA-S6K2-expressing cells showed no increase in DNA synthesis compared to V-293Tet cells. However, Bcl-X_{L} and XIAP expression were selectively increased in KA-S6K2 cells. This was not further induced by FGF-2, could not be suppressed by selective MEK inhibition with PD098059 and was not seen in either KA-S6K1 or wild-type S6K2 over-expressing cells (FIG. 7D and data not shown). Like the KA-S6K2-over-expressing cells, stimulation of HEK293Tet cells with FGF-2 showed increased survival and Bcl-X_{L} and XIAP expression, but the latter could be blocked by MEK inhibition (FIG. 7E, Suppl FIG. 13C). Taken together, these results suggest that S6K2 but not S6K1 regulates Bcl-X_{L} and XIAP expression and that activated S6K2 is sufficient to reproduce FGF-2-induced pro-survival effects.

### Example 6

### S6K2 but not S6K1 Downregulation Enhances Cell Death and Inhibits Clonogenic Growth

To further support the notion that S6K2 is a critical mediator of cell survival, RNAi was employed to specifically down-regulate S6K isoforms and examined cell death by counting viable cells. First, S6K1 or S6K2 RNAi pSR vectors were transfected into HEK293 cells and selective downregulation of the respective targets was verified by western blotting (FIG. 8A upper panel). Compared to S6K1 (S6K1pSR), downregulation of S6K2 (S6K2pSR) increased cell death by about 2 fold in normal growth conditions (FIG. 8A lower panel). Upon serum withdrawal, background cell death increased in both vector and S6K1 knockdown cells. However, S6K2 knockdown induced more cell death (FIG. 8A lower panel).

In H510 cells S6K1pSR and S6K2pSR also induced specific downregulation of the corresponding protein (FIG. 8B-upper panel). Moreover, while expression of

S6K1pSR had no effect on cell survival as compared to empty vector control (pSR), S6K2 downregulation increased basal cell death by greater than two fold over control (FIG. 8B lower panel). This correlated with an increase in the cleavage of lamin B, a substrate of caspase 3 and 7 (FIG. 8B lower panel). In addition, S6K2pSR-H510, unlike the pSR- or S6K1pSR-H510 cells, could not be propagated in culture due to cell death (data not shown). These results support our earlier findings that S6K2 but not S6K1 plays a crucial role in promoting cell survival.

As an additional approach to examine the specific effects of S6K2, clonogenic assays were employed, which at least in part reflect cell survival. FIG. 8C demonstrates that only RNAi-mediated knockdown of S6K2 and not S6K1 expression inhibited the clonogenic growth of HEK293 cells. Similar findings were seen with PKCε knockdown by shRNAi (FIG. 8C). Importantly, these results were not specific to HEK293 cells but could be reproduced in A549 human non-SCLC (NSCLC) and in MCF7 human breast carcinoma cell lines (FIG. 8C). The effect of S6K2 downregulation was also not species specific, as downregulation of S6K2 in NIH 3T3 murine fibroblasts using the same vector (which targets a conserved sequence) led to a decrease of clonogenic growth (FIG. 8C). In contrast, the PKCs targeting sequences used here, that are not conserved between human and mouse, did not reduce NIH 3T3 cell PKCs levels or clonogenic growth (FIG. 8C and data not shown). However, the importance of PKCε in mediating pro-survival effects in murine cells was seen in the KO+ε cells which survived serum withdrawal much better than the KO cells (FIG. 8D). Taken together, these data show that S6K2, but not S6K1, promotes cell survival of HEK293 and H510 SCLC cells and might be widely involved in regulating mammalian cell clonogenic growth.

### Example 7

### S6K2 but not S6K1 Downregulation Inhibits the Anti-Apoptotic Effects of FGF-2

The preceding results suggest that S6K2 mediates the pro-survival effects of FGF-2. To substantiate this, S6K1 and 2 were targeted in H510 cells using the retroviral RNAi vectors described above and subjected the resulting cell lines to etoposide treatment with or without FGF-2. Empty vector (pSR) and S6K1-downregulated (S6K1pSR) H510 cells underwent an equivalent amount of cell death in response to etoposide and were both rescued by pre-incubation with FGF-2 (FIG. 9A). As previously described (Pardo et al., 2002; Pardo et al., 2003), this rescue was mirrored by an increase in Bcl-X_{L} and XIAP protein levels (FIG. 9B). In contrast, H510 cells knocked-down for S6K2 (S6K2pSR) demonstrated a higher background death rate and were almost entirely depleted by etoposide (FIG. 9A). Moreover, pre-treatment with FGF-2 failed to upregulate XIAP or Bcl-X_{L} and could not rescue these cells from etoposide killing (FIGS. 9A and B). These results support the hypothesis that S6K2 mediates FGF-2-induced chemoresistance.

However, the overwhelming cell death induced by etoposide as well as the elevated background death rate in the H510 S6K2pSR cells, could interfere with the ability to observe the pro-survival effects of FGF-2. Therefore, these experiments were repeated using pooled siRNA's, targeting sequences within B-Raf, S6K2 or S6K1 distinct from those recognised by the pSR shRNAi's. FIG. 9C (upper panel) shows that, B-Raf,

S6K2 and S6K1 were selectively downregulated in H510 cells with the respective pooled siRNA's similar to results seen in HEK293 cells (FIG. 12A). Moreover, transient downregulation of S6K2 completely blocked FGF-2-triggered rescue from etoposide killing (FIG. 9C, lower panel). Similar results were obtained with the B-Raf siRNA (FIG. 9C, lower panel). In contrast, transient knockdown of S6K1 failed to block FGF-2-induced chemoresistance. Similar results were seen when these experiments were repeated using individual siRNA to distinct target sequences (FIG. 14). Thus, B-Raf, S6K2 but not S6K1 are required for FGF-2 to provide pro-survival signals that prevent etoposide killing in H510 SCLC cells.

To demonstrate the importance of S6K2 and B-Raf in FGF-2-induced prosurvival signalling in a distinct cell system. The same pSR constructs were transiently transfected in HEK293 cells followed by serum deprivation in the presence or absence of FGF-2. In addition, RNAi vectors for PKCε, PKCα and S6K1 were tested in parallel. While similar amounts of cell death were induced by serum deprivation of pSR and S6K2pSR cells, FGF-2 increased the survival of pSR cells alone (FIG. 9D). Similarly, FGF-2 failed to rescue cells downregulated for B-Raf or PKCs, the two proteins shown to interact with S6K2. In contrast, FGF-2 completely rescued HEK293 cells knocked-down for PKCα (FIG. 9D). Intriguingly, these cells demonstrated a high basal survival rate in the absence of serum and FGF-2. This could potentially be explained by an increase in PKCε phosphorylation at S729, a site linked to its kinase activity (FIG. 9E). Alternatively, PKCα might be required for the induction of cell death in HEK293 cells. Surprisingly, S6K1 downregulation resulted in cell death levels comparable to those observed in pSR cells treated with FGF-2 (FIG. 9D). This might reflect involvement of S6K1 in the induction of cell death. However, downregulation of S6K1 enhanced phosphorylation of S6K2 on S401, a site known to correlate with S6K2 activity, similar to that seen in response to FGF-2 stimulation (FIG. 9F). In contrast, the basal phosphorylation levels of S401 were greatly reduced in the presence of the RNAi targeting S6K2 when compared to control pSR cells (FIG. 9F). Thus, downregulation of S6K1 probably increases S6K2 basal activity mimicking the pro-survival effects induced by FGF-2. Moreover, this likely explains why the addition of FGF-2 fails to further improve the survival of cells knocked-down for S6K1 since these cells already have activated S6K2. Taken together, these data demonstrate that S6K2 is the mediator of FGF-2 prosurvival effects. They, also reveal the non-overlapping roles of S6K1 and S6K2 in cell survival.

The inventors have found that PKCε is both necessary and sufficient to couple FGFRs to MEK/ERK, Bcl-X_{L} and XIAP upregulation and pro-survival effects in both SCLC and HEK 293 cells. Thus, (1) comparison of PKC family member expression levels in a panel of SCLC cell lines revealed that only PKCε correlated with the ability of FGF-2 to induce MEK/ERK signalling and upregulation of Bcl-X_{L} and XIAP, (2) over-expression of PKCs was sufficient to induce MEK/ERK signalling, upregulation of Bcl-X_{L} and XIAP and pro-survival effects, (3) selective suppression of PKCε function or expression prevented these FGF-2-induced effects.

The mechanism by which PKCe might couple to MEK/ERK signalling has been previously investigated. In endothelial cells, PKCε and PKCα can be co-immunprecipitated with Raf-1 in stress mediated MEK/ERK activation (Cheng et al., 2001). In NIH-3T3 cells, PKCε can reside in a latent and inactive complex with Raf-1, which can be stimulated by phorbol ester to trigger MEK/ERK signalling (Hamilton et al., 2001). In contrast to these reports, the inventors were unable to demonstrate a complex between PKCε or PKCα with Raf-1 in either SCLC or HEK293 cells. However, results showed that FGF-2 inducibly triggers the association of PKCe with B-Raf (FIGS. 5, 6 and 12). Moreover, downregulation of B-Raf with various selective RNAi species blocks FGF-2-induced MEK/ERK signalling and pro-survival effects (FIGS. 9, 12 and 14).

FGF-2-induced MEK/ERK signalling, which is necessary for pro-survival effects (Pardo et al., 2002; Pardo et al., 2003), is also required for the activation of S6K2 but not S6K1 (Pardo et al., 2001). Several other findings suggested that S6K2 might have discrete functions as discussed in the introduction. Thus we postulated that S6K2 might associate with the PKCε/BRaf complex and mediate chemoresistance.

These novel findings show that S6K2, but not S6K1, does indeed associate with the FGF-2-induced PKCε/B-Raf complex, a finding common to both SCLC, HEK293 and MEF cells. Intriguingly, it was not possible to reproducibly show the presence of either MEK or ERK in this complex perhaps because the association was weak, transient or blocked antibody recognition. Nevertheless, RNAi knockdown studies in intact cells and co-association studies of purified kinases in vitro indicate that the association of PKCs with S6K2 is direct and results in phosphorylation of S6K2. In contrast, B-Raf only associates with S6K2 in the presence of PKCs in intact cells and cannot directly phosphorylate S6K2 in the absence of PKCs. However, incubation of all three enzymes together further enhances the phosphorylation of S6K2 raising the possibility that B-Raf might phosphorylate S6K2 when PKCs is present. Alternatively, B-Raf might alter the conformation of PKCε and/or S6K2 providing further PKC sites on S6K2. A recent report examining phorbol ester stimulated HEK293 cells suggest that S486 within the C-terminal domain of S6K2 is likely to be one of the PKC regulated sites (Valovka et al., 2003). Clearly, the nature of the S6K2 phosphorylation sites regulated by PKCε and/or B-Raf following physiological stimulation with FGF-2 now warrants further investigation. Regardless of the nature of these sites, though, our results for selective over-expression or inhibition of PKCε function or expression indicate that this kinase mediates FGF-2 induced S6K2 activation (FIG. 5).

Using tetracycline inducible kinase active mutants of S6K2 and S6K1 it is demonstrated that only S6K2 triggers the upregulation of XIAP and Bcl-X_{L} and induced pro-survival effects in HEK293 cells (FIG. 7). Moreover, RNAi knockdown studies in both HEK293 and SCLC cells shows that downregulation of S6K2 but not S6K1 prevents survival (FIG. 8, FIG. 13). In addition, S6K2 is also important for supporting clonogenic growth in several different cell lines (FIG. 8). Crucially, the selective downregulation of S6K2 but not S6K1 blocks FGF-2-induced upregulation of Bcl-X_{L}, XIAP in both SCLC and HEK293 cells and also inhibits death in response to etoposide and serum withdrawal, respectively (FIG. 9, FIG. 14). Thus, S6K2 is both necessary and sufficient to mediate FGF-2-induced pro-survival signalling.

Intriguingly, we have recently found that increased protein expression levels of S6K2 in both SCLC and NSCLC biopsies appears to correlate with the development of chemoresistance (FIG. 15).

A novel FGF-2-induced signalling complex comprising PKCε/BRaf and S6K2 but excluding S6K1. The formation of this complex may explain how S6K1 and S6K2 can be guided to different cellular compartments to target distinct substrates despite their high homology within the kinase domains. Indeed, this complex, via S6K2 (but not S6K1), upregulates Bcl-X_{L} and XIAP protein expression thereby promoting survival/chemoresistance. Thus, the discrete function of S6K2 as opposed to S6K1, has been revealed. Further investigation of the molecular mechanisms by which S6K2 might selectively interact with the translational machinery of the cell to differentially control a subset of anti-apoptotic proteins is now required. Importantly, the targeting of individual members of the PKCε/BRaf/S6K2 signalling complex or their associations could enable the development of novel therapeutic strategies to reverse chemoresistance. Moreover, expression levels of S6K2 and possibly other members of the complex may also provide novel prognostic biomarkers.

### REFERENCES

Calipel, A., Lefevre, G., Pouponnot, C., Mouriaux, F., Eychene, A. and Mascarelli, F. (2003) Mutation of B-Raf in human choroidal melanoma cells mediates cell proliferation and transformation through the MEK/ERK pathway. J Biol Chem, 278, 42409-42418.
Cenni, V., Doppler, H., Sonnenburg, E. D., Maraldi, N., Newton, A. C. and Toker, A. (2002) Regulation of novel protein kinase C epsilon by phosphorylation. Biochem J, 363, 537-545.
Cheng, J. J., Wung, B. S., Chao, Y. J. and Wang, D. L. (2001) Sequential activation of protein kinase C (PKC)-alpha and PKC-epsilon contributes to sustained Raf/ERK1/2 activation in endothelial cells under mechanical strain. J Biol Chem, 276, 31368-31375.
Davies, S. P., Reddy, H., Caivano, M. and Cohen, P. (2000) Specificity and mechanism of action of some commonly used protein kinase inhibitors. Biochem J, 351, 95-105.
Dillon, T. J., Karpitski, V., Wetzel, S. A., Parker, D. C., Shaw, A. S. and Stork, P. J. (2003) Ectopic B-Raf expression enhances extracellular signal-regulated kinase (ERK) signaling in T cells and prevents antigen-presenting cell-induced energy. J Biol Chem, 278, 35940-35949.
Ding, L., Wang, H., Lang, W. and Xiao, L. (2002) Protein kinase C-epsilon promotes survival of lung cancer cells by suppressing apoptosis through dysregulation of the mitochondrial caspase pathway. J Biol Chem, 277, 35305-35313.
Dufner, A. and Thomas, G. (1999) Ribosomal S6 kinase signaling and the control of translation. Exp Cell Res, 253, 100-109.
Erhardt, P., Schremser, E. J. and Cooper, G. M. (1999) B-Raf inhibits programmed cell death downstream of cytochrome c release from mitochondria by activating the MEK/Erk pathway. Mol Cell Biol, 19, 5308-5315.
Gout, I., Minami, T., Hara, K., Tsujishita, Y., Filonenko, V., Waterfield, M. D. and Yonezawa, K. (1998) Molecular cloning and characterization of a novel p70

S6 kinase, p70 S6 kinase β containing a proline-rich sequence. J Biol Chem, 273, 30061-30064.
Gschwendt, M., Muller, H. J., Kielbassa, K., Zang, R., Kittstein, W., Rincke, G. and Marks, F. (1994) Rottlerin, a novel protein kinase inhibitor. Biochem Biophys Res Commun, 199, 93-98.
Hamilton, M., Liao, J., Cathcart, M. K. and Wolfman, A. (2001) Constitutive association of c-N-Ras with c-Raf-1 and protein kinase C epsilon in latent signaling modules. J Biol Chem, 276, 29079-29090.
Ivaska, J., Whelan, R. D., Watson, R. and Parker, P. J. (2002) PKC epsilon controls the traffic of betal integrins in motile cells. Embo J, 21, 3608-3619.
Kawauchi, K., Lazarus, A. H., Sanghera, J. S., Man, G. L., Pelech, S. L. and Delovitch, T. L. (1996) Regulation of BCR- and PKC/Ca(2+)-mediated activation of the Raf1/MEK/MAPK pathway by protein-tyrosine kinase and -tyrosine phosphatase activities. Mol Immunol, 33, 287-296.
Kermorgant, S., Zicha, D. and Parker, P. J. (2004) PKC controls HGF-dependent c-Met traffic signalling and cell migration. EMBO J, 23, 3721-3734.
Lee-Frumen, K. K., Kuo, C. J., Lippincott, J., Terada, N. and Blenis, J. (1999) Characterization of S6K2, a novel kinase homologous to S6K1. Oncogene, 18, 5108-5114.
Martin, K. A., Schlam, S. S., Romanelli, A., Keon, K. L. and Blenis, J. (2001) Ribosomal S6 kinase 2 inhibition by a potent C-terminal repressor domain is relieved by mitogen-activated protein-extracellular signal-related kinase kinase regulated phosphorylation. J. Biol. Chem, 276, 7892-7898.
Pardo, O. E., Arcaro, A., Salerno, G., Raguz, S., Downward, J. and Seckl, M. J. (2002) Fibroblast growth factor-2 induces translational regulation of Bcl-XL and Bcl-2 via a MEK-dependent pathway: correlation with resistance to etoposide-induced apoptosis. J Biol Chem, 277, 12040-12046.
Pardo, O. E., Arcaro, A., Salerno, G., Tetley, T. D., Valovka, T., Gout, I. and Seckl, M. J. (2001) Novel cross talk between MEK and S6K2 in FGF-2 induced proliferation of SCLC cells. Oncogene, 20, 7658-7667.
Pardo, O. E., Lesay, A., Arcaro, A., Lopes, R., Ng, B. L., Warne, P. H., McNeish, I. A., Tetley, T. D., Lemoine, N. R., Mehmet, H., Seckl, M. J. and Downward, J. (2003) Fibroblast growth factor 2-mediated translational control of IAPs blocks mitochondrial release of Smac/DIABLO and apoptosis in small cell lung cancer cells. Mol Cell Biol, 23, 7600-7610.
Parekh, D., Ziegler, W., Yonezawa, K., Hara, K. and Parker, P. J. (1999) Mammalian TOR controls one of two kinase pathways acting upon nPKCdelta and nPKCepsilon. J Biol Chem, 274, 34758-34764.
Pende, M., Urn, S. H., Mieulet, V., Sticker, M., Goss, V. L., Mestan, J., Mueller, M., Fumagalli, S., Kozma, S. C. and Thomas, G. (2004) S6K1(−/−)/S6K2(−/−) mice exhibit perinatal lethality and rapamycin-sensitive 5′-terminal oligopyrimidine mRNA translation and reveal a mitogen-activated protein kinase-dependent S6 kinase pathway. Mol Cell Biol, 24, 3112-3124.
Peraldi, P., Frodin, M., Banner, J. V., Calleja, V., Scimeca, J. C., Filloux, C., Calothy, G. and Van Obberghen, E. (1995) Regulation of the MAP kinase cascade in PC12 cells: B-Raf activates MEK-1 (MAP kinase or ERK kinase) and is inhibited by cAMP. FEBS Lett, 357, 290-296.
Richardson, C. J., Broenstrup, M., Fingar, D. C., Julich, K., Ballif, B. A., Gygi, S. and Blenis, J. (2004) SKAR is a specific target of S6 kinase 1 in cell growth control. Curr Biol, 14, 1540-1549.
Ruotsalainen, T., Joensuu, H., Mattson, K. and Salven, P. (2002) High pretreatment serum concentration of basic fibroblast growth factor is a predictor of poor prognosis in small cell lung cancer. Cancer Epidemiol Biomarkers Prey, 11, 1492-1495.
Schonwasser, D. C., Marais, R. M., Marshall, C. J. and Parker, P. J. (1998) Activation of the mitogen-activated protein kinase/extracellular signal-regulated kinase pathway by conventional, novel, and atypical protein kinase C isotypes. Mol Cell Biol, 18, 790-798.
Seufferlein, T. and Rozengurt, E. (1996) Galanin, neurotensin and phorbol esters rapidly stimulate activation of mitogen activated protein kinase in small cell lung cancer cells. Cancer Res, 56, 5758-5764.
Shima, H., Pende, M., Chen, Y., Fumagalli, S., Thomas, G. and Kozma, S. C. (1998) Disruption of the p70(s6k)/p85(s6k) gene reveals a small mouse phenotype and a new functional S6 kinase. Embo J, 17, 6649-6659.
Soh, J. W., Lee, E. H., Prywes, R. and Weinstein, I. B. (1999) Novel roles of specific isoforms of protein kinase C in activation of the c-fos serum response element. Mol Cell Biol, 19, 1313-1324.
Souroujon, M. C. and Mochly-Rosen, D. (1998) Peptide modulators of protein-protein interactions in intracellular signaling. Nat Biotechnol, 16, 919-924.
Tillman, D. M., Izeradjene, K., Szucs, K. S., Douglas, L. and Houghton, J. A. (2003) Rottlerin sensitizes colon carcinoma cells to tumor necrosis factor-related apoptosis-inducing ligand-induced apoptosis via uncoupling of the mitochondria independent of protein kinase C. Cancer Res, 63, 5118-5125.
Valovka, T., Verdier, F., Cramer, R., Zhyvoloup, A., Fenton, T., Rebholz, H., Wang, M. L., Gzhegotsky, M., Lutsyk, A., Matsuka, G., Filonenko, V., Wang, L., Proud, C. G., Parker, P. J. and Gout, I. T. (2003) Protein kinase C phosphorylates ribosomal protein S6 kinase betaII and regulates its subcellular localization. Mol Cell Biol, 23, 852-863.
Vives, E., Brodin, P. and Lebleu, B. (1997) A truncated HIV-1 Tat protein basic domain rapidly translocates through the plasma membrane and accumulates in the cell nucleus. J Biol Chem, 272, 16010-16017.
Wan, P. T., Garnett, M. J., Roe, S. M., Lee, S., Niculescu-Duvaz, D., Good, V. M., Jones, C. M., Marshall, C. J., Springer, C. J., Basford, D. and Marais, R. (2004) Mechanism of activation of the RAF-ERK signaling pathway by oncogenic mutations of B-RAF. Cell, 116, 855-867.
Wang, L., Gout, I. and Proud, C. G. (2001) Cross-talk between the ERK and p70 S6 kinase (S6K) signaling pathways. MEEK-dependent activation of S6K2 in cardiomyocytes. J Biol Chem, 276, 32670-32677.
Way, K. J., Chou, E. and King, G. L. (2000) Identification of PKC-isoform-specific biological actions using pharmacological approaches. Trends Pharmacol Sci, 21, 181-187.
Wu, D., Thakore, C. U., Wescott, G. G., McCubrey, J. A. and Terrian, D. M. (2004) Integrin signaling links protein kinase Cepsilon to the protein kinase B/Akt survival pathway in recurrent prostate cancer cells. Oncogene, 23, 8659-8672.
Yedovitzky, M., Mochly-Rosen, D., Johnson, J. A., Gray, M. O., Ron, D., Abramovitch, E., Cerasi, E. and Nesher, R. (1997) Translocation inhibitors define specificity of protein kinase C isoenzymes in pancreatic beta-cells. J Biol Chem, 272, 1417-1420.
Zou, Y., Komuro, I., Yamazaki, T., Aikawa, R., Kudoh, S., Shiojima, I., Hiroi, Y., Mizuno, T. and Yazaki, Y. (1996) Protein kinase C, but not tyrosine kinases or Ras, plays a critical role in angiotensin II-induced activation of Raf-1 kinase and extracellular signal-regulated protein kinases in cardiac myocytes. J Biol Chem, 271, 33592-33597.

1. A complex comprising two or more of S6K2, B-Raf and PKCε.

2. The complex of claim 1, which is capable of causing chemoresistance in a cancer cell.

3. The complex of claim 1, wherein S6K2 comprises the sequence as set out in FIG. 1.

4. The complex of claim 1, wherein B-Raf comprises the sequence as set out in FIG. 2.

5. The complex of claim 1, wherein PKCε comprises the sequence as set out in FIG. 3.

6. An antibody which binds specifically to the complex of claim 1.

7. A method for identifying an inhibitor of the complex of claim 1, comprising the steps of contacting a cell which expresses two or more of S6K2, PKCε and B-Raf with a test compound and determining whether a complex is formed.

8. A method for identifying an inhibitor of the complex of claim 1, comprising the steps of contacting two or more of S6K2, PKCε and B-Raf with a test compound and determining whether a complex is formed.

9. An inhibitor of the complex of claim 1.

10. The inhibitor of claim 9, which is identified by the method of comprising the steps of contacting a cell which expresses two or more of S6K2, PKCε and B-Raf with a test compound and determining whether a complex is formed.

11. The inhibitor of claim 9, wherein the inhibitor inhibits B-Raf expression.

12. The inhibitor of claim 9, wherein the inhibitor inhibits PKCε expression.

13. The inhibitor of claim 9, wherein the inhibitor inhibits S6K2 expression.

14. The inhibitor of claim 9, wherein the inhibitor prevents the association of S6K2, B-Raf and/or PKCε.

15. A method of preventing or reversing chemoresistance in a cancer cell comprising administering to the cell an inhibitor of the complex of claim 1.

16. The method of claim 15, wherein the inhibitor is identified by the method comprising the steps of contacting a cell which expresses two or more of S6K2, PKCε and B-Raf with a test compound and determining whether a complex is formed.

17. The method according to claim 15, wherein the cancer cell is small cell lung cancer (SCLC) cell.

18. The method according to claim 15, wherein the inhibitor is RNAi, antisense RNA, ribozyme RNA or an antibody.

19. A pharmaceutical composition comprising an inhibitor of claim 14 and a pharmaceutically acceptable adjuvant, diluent or excipient.

20. A method of diagnosing chemoresistance in a cancer patient comprising detecting of a complex comprising S6K2, B-Raf and PKCε in a cancer cell of the patient.

21. A method of diagnosing chemoresistance in a cancer patient comprising detecting the level of S6K2 activation in a cancer cell of the patient and comparing to the level of S6K2 activation in a non-cancer cell of the patient or a cell in a non-cancer patient, wherein the cancer cell is resistant to chemotherapy if the level of S6K2 activation is higher in the cancer cell than in the non-cancer cell or the cell from a non-cancer patient.

22. A method of predicting the likelihood of a cancer cell developing chemoresistance, comprising measuring the level of S6K2 activation in the cell at two or more time points, wherein the cancer cell is likely to develop chemoresistance if the level of S6K2 activation increases between time points.

23. A method of diagnosing chemoresistance in a cancer cell comprising detecting the level of S6K2 in the cell.

24. The use of an inhibitor of the complex of claim 1 in the manufacture of a medicament for the prevention or reversal of chemoresistance in a cancer cell.


SEQUENCE LISTING (34 sequences)

SEQ ID NO: 1 - protein, 482 aa, Homo sapiens
MAAVFDLDLETEEGSEGEGEPELSPADACPLAELRAAGLEPVGHYEEVELTETSVNVGPERIGPHCFELLRVLGKGGYGKVFQVRKVQGTNLGKIYAMKVLRKAKIVRNAKDTAHTRAERNILESVKHPFIVELAYAFQTGGKLYLILECLSGGELFTHLEREGIFLEDTACFYLAEITLALGHLHSQGIIYRDLKPENIMLSSQGHIKLTDFGLCKESIHEGAVTHTFCGTIEYMAPEILVRSGHNRAVDWWSLGALMYDMLTGSPPFTAENRKKTMDKIIRGKLALPPYLTPDARDLVKKFLKRNPSQRIGGGPGDAADVQRHPFFRHMNWDDLLAWRVDPPFRPCLQSEEDVSQFDTRFTRQTPVDSPDDTALSESANQAFLGFTYVAPSVLDSIKEGFSFQPKLRSPRRLNSSPRAPVSPLKFSPFEGFRPSPSLPEPTELPLPPLLPPPPPSTTAPLPIRPPSGTKKSKRGRGRPGR

SEQ ID NO: 2 - protein, 766 aa, Homo sapiens [sequence omitted]

SEQ ID NO: 3 - protein, 737 aa, Homo sapiens
MVVFNGLLKIKICEAVSLKPTAWSLRHAVGPRPQTFLLDPYIALNVDDSRIGQTATKQKTNSPAWHDEFVTDVCNGRKIELAVFHDAPIGYDDFVANCTIQFEELLQNGSRHFEDWIDLEPEGRVYVIIDLSGSSGEAPKDNEERVFRERMRPRKRQGAVRRRVHQVNGHKFMATYLRQPTYCSHCRDFIWGVIGKQGYQCQVCTCVVHKRCHELIITKCAGLKKQETPDQVGSQRFSVNMPHKFGIHNYKVPTFCDHCGSLLWGLLRQGLQCKVCKMNVHRRCETNVAPNCGVDARGIAKVLADLGVTPDKITNSGQRRKKLIAGAESPQPASGSSPSEEDRSKSAPTSPCDQEIKELENNIRKALSFDNRGEEHRAASSPDGQLMSPGENGEVRQGQAKRLGLDEFNFIKVLGKGSFGKVMLAELKGKDEVYAVKVLKKDVILQDDDVDCTMTEKRILALARKHPYLTQLYCCFQTKDRLFFVMEYVNGGDLMFQIQRSRKFDEPRSRFYAAEVTSALMFLHQHGVIYRDLKLDNILLDAEGHCKLADFGMCKEGILNGVTTTTFCGTPDYIAPEILQELEYGPSVDWWALGVLMYEMMAGQPPFEADNEDDLFESILHDDVLYPVWLSKEAVSILKAFMTKNPHKRLGCVASQNGEDAIKQHPFFKEIDWVLLEQKKIKPPFKPRIKTKRDVNNFDQDFTREEPVLTLVDEAIVKQINQEEFKGFSYFGEDLMP

SEQ ID NO: 4 - protein, 8 aa, Artificial
  Translocation inhibitor peptide
EAVSLKPT

SEQ ID NO: 5 - protein, 9 aa, Artificial
  Translocation inhibitor peptide
SLNPEWNET

SEQ ID NO: 6 - protein, 13 aa, Human immunodeficiency virus
GRKKRRQRRRPPQ

SEQ ID NO: 7 - DNA, 19 bp, Artificial
  pSUPER Retro construct
caaggcttccagtgccaag

SEQ ID NO: 8 - DNA, 19 bp, Artificial
  pSUPER Retro constructs
ggaacacatgatggatgga

SEQ ID NO: 9 - DNA, 19 bp, Artificial
  pSUPER Retro construct
catggaactcaggcagaaa

SEQ ID NO: 10 - DNA, 19 bp, Artificial
  pSUPER Retro construct
tctgcgaggccgtgagctt

SEQ ID NO: 11 - DNA, 19 bp, Artificial
  pSUPER Retro construct
ctacaaggtccctaccttc

SEQ ID NO: 12 - DNA, 19 bp, Artificial
  pSUPER Retro construct
ggaagggattctgaatggt

SEQ ID NO: 13 - DNA, 19 bp, Artificial
  pSUPER Retro construct
ggttctgggccagggatcc

SEQ ID NO: 14 - DNA, 19 bp, Artificial
  pSUPER Retro construct
gctctatctcattctggac

SEQ ID NO: 15 - DNA, 19 bp, Artificial
  pSUPER Retro construct
catcatcactctgaaagat

SEQ ID NO: 16 - DNA, 19 bp, Artificial
  pSUPER Retro construct
ggggggctatggcaaggtg

SEQ ID NO: 17 - DNA, 19 bp, Artificial
  pSUPER Retro construct
cggaatcccagccagcgga

SEQ ID NO: 18 - DNA, 19 bp, Artificial
  pSUPER Retro construct
gatacggcctgcttctacc

SEQ ID NO: 19 - DNA, 19 bp, Artificial
  pSUPER Retro construct
caacagttattggaatctc

SEQ ID NO: 20 - DNA, 19 bp, Artificial
  pSUPER Retro construct
cctatcgttagagtcttcc

SEQ ID NO: 21 - DNA, 19 bp, Artificial
  pSUPER Retro construct
gaattggatctggatcatt

SEQ ID NO: 22 - DNA, 19 bp, Artificial
  pSUPER Retro construct
cagtggtcaatgtgcgaaa

SEQ ID NO: 23 - DNA, 19 bp, Artificial
  pSUPER Retro construct
gaacttcaagtagatttcc

SEQ ID NO: 24 - DNA, 19 bp, Artificial
  pSUPER Retro construct
catcagacaactcttattg

SEQ ID NO: 25 - RNA, 21 nt, Artificial
  siRNA
gcaaggagucuauccaugauu

SEQ ID NO: 26 - RNA, 21 nt, Artificial
  siRNA
gacgugagccaguuugauauu

SEQ ID NO: 27 - RNA, 21 nt, Artificial
  siRNA
ggaagaaaaccauggauaauu

SEQ ID NO: 28 - RNA, 21 nt, Artificial
  siRNA
ggaacauucuagagucaguuu

SEQ ID NO: 29 - RNA, 22 nt, Artificial
  siRNA
nacugacucuagaauguuccuu

SEQ ID NO: 30 - RNA, 19 nt, Artificial
  siRNA
gcaggaguguuugacauag

SEQ ID NO: 31 - RNA, 19 nt, Artificial
  siRNA
gacaaaauccucaaaugua

SEQ ID NO: 32 - RNA, 19 nt, Artificial
  siRNA
cauggaacauugugagaaa

SEQ ID NO: 33 - RNA, 19 nt, Artificial
  siRNA
ccaaggucaugugaaacua

SEQ ID NO: 34 - RNA, 21 nt, Artificial
  Oligonucleotide targetting sequence
aaagaauuggaucuggaucau